(19) United States
(12) Patent Application Publication
(10) Pub. No.: US 2026/0027124 A1
(43) Pub. Date: Jan. 29, 2026

(54) SUBSTITUTED IMIDAZO[1,2-A]PYRIDINE COMPOUNDS AND THE USE THEREOF IN THE TREATMENT AND PREVENTION OF FIBROSIS

(71) Applicant: MOEXA PHARMACEUTICALS LIMITED, Hong Kong (CN)

(72) Inventors: Ho Yin LO, Hong Kong (CN); Robert Stephen PORTER, Hong Kong (CN)

(21) Appl. No.: 18/996,898

(22) PCT Filed: Jul. 14, 2023
(86) PCT No.: PCT/CN2023/107345

§ 371 Date: Jan. 17, 2025

Related U.S. Application Data
(60) Provisional application No. 63/390,227, filed on Jul. 18, 2022.

(51) Int. Cl. A61K 31/5377; A61K 31/437; A61K 31/4375; A61K 31/4545; A61K 31/4725; A61K 31/496; A61K 31/497; A61K 31/506; A61K 45/06; C07D 471/04; C07D 519/00

(52) U.S. Cl. CPC A61K 31/5377; A61K 31/437; A61K 31/4375; A61K 31/4545; A61K 31/4725; A61K 31/496; A61K 31/497; A61K 31/506; A61K 45/06; C07D 471/04; C07D 519/00

(57) ABSTRACT

Provided are a class of substituted imidazo[1,2-a]pyridine compounds, pharmaceutical compositions containing the same, and use of the compounds in the treatment and prevention of fibrosis.

### TECHNICAL FIELD

The present disclosure relates generally to a class of substituted imidazo[1,2-a]pyridine compounds, compositions containing the same and the therapeutic use of the compounds in the treatment and prevention of fibrosis.

### BACKGROUND ART

Fibrosis is the development of fibrous connective tissue as a reparative response to injury or damage. Pulmonary fibrosis refers to a number of conditions that cause interstitial lung damage followed by fibrosis, and eventually loss of lung elasticity. These conditions lead to symptoms such as persistent cough, chest pain, difficulty breathing and fatigue. The average life expectancy of patients with pulmonary fibrosis is three to five years after diagnosis. As such, early detection and treatment is the key to slowing progression and prolonging life.

In December 2019 reports emerged of a severe acute respiratory disease caused by severe acute respiratory syndrome coronavirus 2. The virus has been named “Covid-19”. By the end of July 2020, over 20 million people globally had been infected with Covid-19 and there have been more than 750,000 deaths.

Recent reports have noted many instances of pulmonary fibrotic disease occurring in subjects diagnosed with Covid-19, ranging from fibrosis associated with pneumonia to severe acute lung injury, in which there is widespread fibrotic change. It has also been observed that in fatal cases of Covid-19 pulmonary fibrosis is typically present at autopsy, with anecdotal reports of severe fibrotic organising pneumonia. Previous coronavirus outbreaks (such as severe acute respiratory syndrome (SARS) in 2003 and Middle East respiratory syndrome (MERS) have been associated with substantial post-viral fibrosis and physiological impairment. Given the large scale of the current Covid-19 pandemic it is likely that the burden of fibrotic lung disease will increase dramatically and remain a significant problem for many years to come. The risk of future viral outbreaks caused by other respiratory viruses (for example those of avian and swine origin) has the potential to further increase the burden.

There is therefore an urgent need for new therapeutic and preventative options for treating and preventing fibrosis, and in particular fibrosis that is associated with Covid-19.

### SUMMARY OF THE INVENTION

In a first aspect there is provided a method for treating fibrosis in a subject in need thereof, the method comprising administration to the subject of a therapeutically effective amount of a compound having the following formula (I), or a pharmaceutically acceptable salt or prodrug thereof:

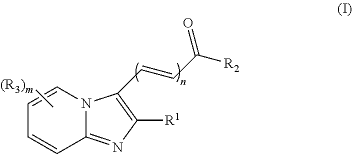

wherein:

R₁ is selected from the group consisting of: C₆₋₁₀aryl, a heteroaryl group having between 5 and 10 ring atoms in which one or more of the ring atoms are selected from nitrogen, sulfur and oxygen, C₃₋₁₀cycloalkyl and a heterocyclyl group having between 5 and 10 ring atoms in which one or more of the ring atoms are selected from nitrogen, sulfur and oxygen, each of which is optionally substituted with one or more of the following groups: C₁₋₆alkoxy, C₁₋₆alkyl, CN, halo and C₃₋₁₀cycloalkyl;
R₂ is selected from the group consisting of:
(i) C₆₋₁₀aryl, a heteroaryl group having between 5 and 10 ring atoms in which one or more of the ring atoms are selected from nitrogen, sulfur and oxygen, and a heterocyclyl group having between 5 and 10 ring atoms in which one or more of the ring atoms are selected from nitrogen, sulfur and oxygen, each of which is optionally substituted with one or more of the following groups: NR₅R₆, C₁₋₆alkyl, C₁₋₆alkoxy and halo; and
(ii)-X-Y,
in which X is selected from the group consisting of: a heterocyclyl group having between 5 and 10 ring atoms in which one or more of the ring atoms are selected from nitrogen, sulfur and oxygen, C₆₋₁₀aryl and a heteroaryl group having between 5 and 10 ring atoms in which one or more of the ring atoms are selected from nitrogen, sulfur and oxygen; and Y is selected from the group consisting of: C₆₋₁₀aryl and a heteroaryl group having between 5 and 10 ring atoms in which one or more of the ring atoms are selected from nitrogen, sulfur and oxygen, and wherein the C₆₋₁₀aryl and heteroaryl groups of Y are optionally substituted with one or more of the following groups: NR₅R₆, C₁₋₆alkyl, C₁₋₆alkoxy and halo;
R₃ is selected from the group consisting of: halo, C₁₋₆ alkoxy, OH, CN, NO₂, —OC(O)R₄, —C(O)R₄, —C(O)NR₅R₆, —C(O)OR₄, —OS(O)₂R₄, NR₅R₆, SR₄, C₁₋₁₀alkyl, C₂₋₁₀alkenyl, C₂₋₁₀alkynyl, C₃₋₁₀cycloalkyl, C₆₋₁₀aryl and a heteroaryl group having between 5 and 10 ring atoms in which one or more of the ring atoms are selected from nitrogen, sulfur and oxygen;
R₄ is selected from H and C₁₋₆alkyl;
R₅ and R₆ are independently selected from H and C₁₋₆alkyl;
n is 1 or 2; and,
m is an integer between 0 and 4.

The fibrosis may be pulmonary fibrosis. In one embodiment, the fibrosis is idiopathic pulmonary fibrosis.

The method may reduce fibrotic damage.

The fibrosis may be caused by, associated with, or otherwise related to a coronavirus infection in the subject.

The subject may be suffering from a coronavirus infection, or the subject may have recovered from a coronavirus infection.

The corona virus may be a severe acute respiratory syndrome coronavirus 2 (SARS-CoV-2), such as for example Covid-19.

The compound of formula (I) may be administered together with one or more anti-inflammatory compounds.

The compound of formula (I) may be administered together with one or more antiviral compounds.

In a second aspect the present invention provides a method for preventing or minimising fibrosis in a subject, wherein the subject is infected with a coronavirus, or at risk of becoming infected with a coronavirus, the method comprising administering to the subject an effective amount of a compound of formula (I) as defined in the first aspect.

The subject may be suffering from pneumonia and/or acute respiratory distress syndrome (ARDS).

The compound of formula (I) may be administered within a week of the subject being diagnosed with the coronavirus.

The compound of formula (I) may be administered within a week of onset of ARDS in the subject.

The coronavirus may be Covid-19.

The method may prevent or minimise fibrotic damage.

The compound of formula (I) may be administered together with one or more anti-inflammatory compounds.

The compound of formula (I) may be administered together with one or more antiviral compounds.

In a third aspect the present invention provides a method for preventing or minimising lung injury or damage in a subject, wherein the subject is infected with a coronavirus, or at risk of becoming infected with a coronavirus, the method comprising administering to the subject an effective amount of a compound of formula (I) as defined in the first aspect.

The coronavirus may be Covid-19.

The lung injury or damage may be caused by, associated with, or otherwise related to fibrosis.

The compound of formula (I) may be administered together with one or more anti-inflammatory compounds.

The compound of formula (I) may be administered together with one or more antiviral compounds.

In a fourth aspect the present invention provides a method for minimising the severity of a coronavirus infection in a subject, the method comprising administering to the subject an effective amount of a compound of formula (I) as defined in the first aspect.

The coronavirus may be Covid-19.

The compound of formula (I) may be administered together with one or more anti-inflammatory compounds.

The compound of formula (I) may be administered together with one or more antiviral compounds.

In a fifth aspect there is provided a compound of formula (II), or a pharmaceutically acceptable salt or prodrug thereof:

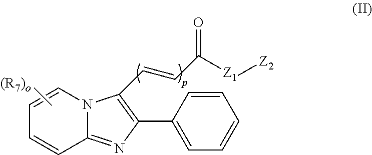

wherein:
R₇ is selected from the group consisting of: halo, OC₁₋₆ alkyl, OH, CN, NO₂, —OC(O)R₄, —C(O)R₄, —C(O)NR₅R₆, —C(O)OR₄, —OS(O)₂R₄, NR₅R₆, SR₄, C₁₋₁₀alkyl, C₂₋₁₀alkenyl, C₂₋₁₀alkynyl; C₃₋₁₀cycloalkyl, C₆₋₁₀aryl and a heteroaryl group having between 5 and 10 ring atoms in which one or more of the ring atoms are selected from nitrogen, sulfur and oxygen;
Z₁ is a heterocyclyl group having between 5 and 10 ring atoms in which one or more of the ring atoms are selected from nitrogen, sulfur and oxygen;
Z₂ is selected from: phenyl, a heteroaryl group having between 5 and 10 ring atoms in which one or more of the ring atoms are selected from nitrogen, sulfur and oxygen, and a heterocyclyl group having between 5 and 10 ring atoms in which one or more of the ring atoms are selected from nitrogen, sulfur and oxygen, each of which is optionally substituted with one or more of the following groups: NR₅R₆, C₁₋₆alkyl, OC₁₋₆alkyl and halo;
p is 1 or 2; and,
o is an integer between 0 and 4,
and wherein R₄ to R₆ are as defined in the first aspect.

In a sixth aspect there is provided a pharmaceutical composition comprising a compound of formula (II) according to the fifth aspect together with a pharmaceutically acceptable carrier, diluent or excipient.

In a seventh aspect there is provided use of a compound of formula (I) as defined in the first aspect in the manufacture of medicament for the treatment of fibrosis.

In an eighth aspect there is provided use of a compound of formula (I) as defined in the first aspect in the manufacture of a medicament for preventing or minimising fibrosis in a subject, wherein the subject is infected with a coronavirus, or at risk of becoming infected with a coronavirus.

In a ninth aspect there is provided use of a compound of formula (I) as defined in the first aspect in the manufacture of a medicament for preventing or minimising lung injury or damage in a subject, wherein the subject is infected with a coronavirus, or at risk of becoming infected with a coronavirus.

In a tenth aspect there is provided use of a compound of formula (I) as defined in the first aspect in the manufacture of a medicament for minimising the severity of a coronavirus infection.

In an eleventh aspect there is provided a compound of formula (I) as defined in the first aspect for use in a method for the treatment of fibrosis.

In a twelfth aspect there is provided a compound of formula (I) as defined in the first aspect for use in a method for preventing or minimising fibrosis in a subject, wherein the subject is infected with a coronavirus, or at risk of becoming infected with a coronavirus.

In a thirteenth aspect there is provided a compound of formula (I) as defined in the first aspect for use in a method for preventing or minimising lung injury or damage in a subject, wherein the subject is infected with a coronavirus, or at risk of becoming infected with a coronavirus.

In a fourteenth aspect there is provided a compound of formula (I) as defined in the first aspect for use in a method for minimising the severity of a coronavirus infection.

### Definitions

The following are some definitions that may be helpful in understanding the description of the present disclosure. These are intended as general definitions and should in no way limit the scope of the present disclosure to those terms alone, but are put forth for a better understanding of the following description.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The terms “a” and “an” are used herein to refer to one or to more than one (i.e. to at least one) of the grammatical object of the article. By way of example, “an element” means one element or more than one element.

In the context of this specification, the term “alkyl” is taken to mean straight-chain or branched-chain monovalent saturated hydrocarbon groups having the recited number of carbon atoms. Examples of alkyl groups include, but are not limited to, methyl, ethyl, 1-propyl, isopropyl, 1-butyl, 2-butyl, isobutyl, tert-butyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl and the like.

In the context of this specification, the term “alkenyl” is taken to mean straight chain or branched chain monovalent hydrocarbon radicals having the recited number of carbon atoms and at least one carbon-carbon double bond, such as vinyl, propenyl, 2-methyl-2-propenyl, butenyl and the like. The group may contain a plurality of double bonds and the geometry about each double bond is independently cis or trans, E or Z.

In the context of this specification, the term “alkynyl” is taken to mean straight chain or branched chain monovalent hydrocarbon radicals having the recited number of carbon atoms and at least one carbon-carbon triple bond, such as ethynyl, propargyl and the like. The group may contain a plurality of triple bonds.

In the context of this specification, the term “alkoxy” is taken to mean O-alkyl groups in which alkyl is as defined herein. Examples of alkoxy groups include, but are not limited to, methoxy, ethoxy, n-propoxy, isopropoxy, sec-butoxy and tert-butoxy.

In the context of this specification, the terms “halo” and “halogen” are used interchangeably and refer to fluorine, chlorine, bromine and iodine.

In the context of this specification, the term “aryl” is taken to mean monovalent aromatic hydrocarbon groups having the recited number of ring carbon atoms. The aryl group may have a single ring or multiple rings. “Aryl” also includes bicyclic radicals comprising an aromatic ring fused to a saturated or partially unsaturated carbocyclic ring. Examples of aryl groups include, but are not limited to, phenyl, naphthyl, phenanthrenyl, azulenyl, anthracenyl, 1,2-dihydronaphthyl, 1,2,3,4-tetrahydronaphthyl and the like.

In the context of this specification, the term “heteroaryl” refers to a monocyclic, bicyclic, or tricyclic aromatic ring system having the recited total number of ring atoms, wherein the ring system contains at least one nitrogen, sulfur or oxygen atom, the remaining ring atoms being carbon. Examples of heteroaryl include, but are not limited to pyridyl, pyrrolyl, indolyl, quinolinyl, furnayl, thienyl, oxazolyl, thiazolyl and the like.

In the context of this specification, the term “heterocyclyl” refers to a non-aromatic, saturated or partially unsaturated ring system having the recited total number of ring atoms, wherein the ring system contains at least one nitrogen, sulfur or oxygen atom, the remaining ring atoms being carbon. The term also includes substituents in which the heterocyclyl group is fused with an aromatic ring. Examples of heterocyclyl include, but are not limited to pyrrolidinyl, piperazinyl, 2,3-dihydroindolyl, piperidinyl, azetidinyl, pyrazolinyl, morpholinyl, dihydroquinolinyl and the like.

In the context of this specification, the term “cycloalkyl” is taken to mean monovalent, saturated carbocyclic rings having the recited number of ring carbon atoms. The ring may be monocyclic or bicyclic. Bicyclic carbocycles may be arranged, for example, as a bicyclo [4,5], [5,5], [5,6] or [6,6], or as bridged systems, such as bicyclo[2.2.1]heptane. Examples of carbocyclyl groups include, but are not limited to, cyclopropyl, cyclobutyl, cyclopentyl, cyclooctyl, cyclodecyl and the like.

In the context of this specification, the term “prodrug” means a compound which is able to be converted in vivo by metabolic means (e.g. by hydrolysis, reduction or oxidation) to a compound of the formulae (I) or (II).

In the context of this specification, the term “effective amount” includes a non-toxic but sufficient amount of an active compound to provide the stated effect. Those skilled in the art will appreciate that the exact amount of a compound required will vary based on a number of factors and thus it is not possible to specify an exact “effective amount”. However, for any given case an appropriate “effective amount” may be determined by one of ordinary skill in the art.

In the context of this specification, the term “therapeutically effective amount” includes a non-toxic but sufficient amount of an active compound to provide the desired therapeutic effect. Those skilled in the art will appreciate that the exact amount of a compound required will vary based on a number of factors and thus it is not possible to specify an exact “therapeutically effective amount”. However, for any given case an appropriate “therapeutically effective amount” may be determined by one of ordinary skill in the art.

In the context of this specification, the terms “treating”, “treatment”, “preventing” and “prevention” refer to any and all uses that remedy a disease state or symptoms thereof, prevent the establishment of a disease state, or otherwise prevent, hinder, retard or reverse the progression of a disease or other undesirable symptoms in any way whatsoever. Thus, the terms “treating”, “treatment”, “preventing” and “prevention” and the like are to be considered in their broadest context. For example, treatment does not necessarily imply that a subject is treated until total recovery.

In the context of this specification, the term “subject” includes human and also non-human animals. As such, in addition to being useful in the treatment of humans, the compounds of the present disclosure also find use in the treatment of non-human animals, for example mammals such as companion animals and farm animals. Non-limiting examples of companion animals and farm animals include dogs, cats, horses, cows, sheep and pigs. Preferably, the subject is a human.

In the context of this specification the term “administering” and variations of that term including “administer” and “administration”, includes contacting, applying, delivering or providing a compound or composition to an organism by any appropriate means.

### DETAILED DESCRIPTION

A new class of substituted imidazo[1,2-a]pyridine compounds that show promise in the treatment and prevention of fibrosis and related lung conditions associated with Covid-19 is described in detail.

In one aspect there is provided a method for treating fibrosis in a subject in need thereof, the method comprising administration to the subject of a therapeutically effective amount of a compound having the following formula (I), or a pharmaceutically acceptable salt or prodrug thereof:

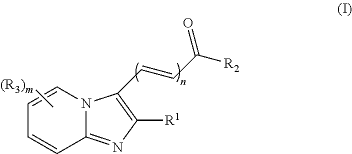

wherein:
R₁ is selected from the group consisting of: C₆₋₁₀aryl, a heteroaryl group having between 5 and 10 ring atoms in which one or more of the ring atoms are selected from nitrogen, sulfur and oxygen, C₃₋₁₀cycloalkyl and a heterocyclyl group having between 5 and 10 ring atoms in which one or more of the ring atoms are selected from nitrogen, sulfur and oxygen, each of which is optionally substituted with one or more of the following groups: C₁₋₆alkoxy, C₁₋₆alkyl, CN, halo and C₃₋₁₀cycloalkyl;
R₂ is selected from the group consisting of:
(i) C₆₋₁₀aryl, a heteroaryl group having between 5 and 10 ring atoms in which one or more of the ring atoms are selected from nitrogen, sulfur and oxygen, and a heterocyclyl group having between 5 and 10 ring atoms in which one or more of the ring atoms are selected from nitrogen, sulfur and oxygen, each of which is optionally substituted with one or more of the following groups: NR₅R₆, C₁₋₆alkyl, C₁₋₆alkoxy and halo; and
(ii)-X-Y,
in which X is selected from the group consisting of: a heterocyclyl group having between 5 and 10 ring atoms in which one or more of the ring atoms are selected from nitrogen, sulfur and oxygen, C₆₋₁₀aryl and a heteroaryl group having between 5 and 10 ring atoms in which one or more of the ring atoms are selected from nitrogen, sulfur and oxygen; and Y is selected from the group consisting of: C₆₋₁₀aryl and a heteroaryl group having between 5 and 10 ring atoms in which one or more of the ring atoms are selected from nitrogen, sulfur and oxygen, and wherein the C₆₋₁₀aryl and heteroaryl groups of Y are optionally substituted with one or more of the following groups: NR₅R₆, C₁₋₆alkyl, C₁₋₆alkoxy and halo;
R₃ is selected from the group consisting of: halo, C₁₋₆ alkoxy, OH, CN, NO₂, —OC(O)R₄, —C(O)R₄, —C(O)NR₅R₆, —C(O)OR₄, —OS(O)₂R₄, NR₅R₆, SR₄, C₁₋₁₀alkyl, C₂₋₁₀alkenyl, C₂₋₁₀alkynyl, C₃₋₁₀cycloalkyl, C₆₋₁₀aryl and a heteroaryl group having between 5 and 10 ring atoms in which one or more of the ring atoms are selected from nitrogen, sulfur and oxygen;
R₄ is selected from H and C₁₋₆alkyl;
R₅ and R₆ are independently selected from H and C₁₋₆alkyl;
n is 1 or 2; and,
m is an integer between 0 and 4.

In another embodiment R₁ is selected from the group consisting of: C₆₋₁₀aryl, a heteroaryl group having between 5 and 10 ring atoms in which one or more of the ring atoms are selected from nitrogen, sulfur and oxygen, C₃₋₁₀cycloalkyl and a heterocyclyl group having between 5 and 10 ring atoms in which one or more of the ring atoms are selected from nitrogen, sulfur and oxygen, each of which is optionally substituted with one or more of the following groups: C₁₋₄alkoxy, C₁₋₄alkyl, CN, halo and C₃₋₆cycloalkyl.

In a further embodiment R₁ is selected from the group consisting of: C₆₋₁₀aryl, a heteroaryl group having between 5 and 10 ring atoms in which one or more of the ring atoms are selected from nitrogen, sulfur and oxygen, C₃₋₆cycloalkyl and a heterocyclyl group having between 5 and 10 ring atoms in which one or more of the ring atoms are selected from nitrogen, sulfur and oxygen, each of which is optionally substituted with one or more of the following groups: C₁₋₄alkoxy, C₁₋₄alkyl, halo and C₃₋₆cycloalkyl.

In yet another embodiment R₁ is selected from the group consisting of: C₆₋₁₀aryl, a heteroaryl group having between 5 and 10 ring atoms in which one or more of the ring atoms are selected from nitrogen, sulfur and oxygen and a heterocyclyl group having between 5 and 10 ring atoms in which one or more of the ring atoms are selected from nitrogen, sulfur and oxygen, each of which is optionally substituted with one or more of the following groups: C₁₋₄alkyl, halo and C₃₋₆cycloalkyl.

In still a further embodiment R₁ is selected from the group consisting of: C₆₋₁₀aryl, a heteroaryl group having 5 or 6 ring atoms in which one or more of the ring atoms are selected from nitrogen, sulfur and oxygen and a heterocyclyl group having 5 or 6 ring atoms in which one or more of the ring atoms are selected from nitrogen, sulfur and oxygen, each of which is optionally substituted with one or more of the following groups: C₁₋₄alkyl, halo and C₃₋₆cycloalkyl.

In another embodiment R₁ is selected from the group consisting of: phenyl, a heteroaryl group having 5 or 6 ring atoms in which one or more of the ring atoms are selected from nitrogen, sulfur and oxygen and a heterocyclyl group having 5 or 6 ring atoms in which one or more of the ring atoms are selected from nitrogen, sulfur and oxygen, each of which is optionally substituted with one or more of the following groups: C₁₋₄alkyl, halo and C₃₋₄cycloalkyl.

In still a further embodiment R₁ is selected from the group consisting of: phenyl, a heteroaryl group having 5 or 6 ring atoms in which one or more of the ring atoms are selected from nitrogen, sulfur and oxygen and a heterocyclyl group having 5 or 6 ring atoms in which one or more of the ring atoms are selected from nitrogen, sulfur and oxygen, each of which is optionally substituted with one or more of the following groups: C₁₋₃alkyl, halo and cyclopropyl.

In yet another embodiment R₁ is selected from the group consisting of: phenyl, a heteroaryl group having 5 or 6 ring atoms in which between one and three of the ring atoms are selected from nitrogen, sulfur and oxygen and a heterocyclyl group having 5 or 6 ring atoms in which one or two of the ring atoms are selected from nitrogen, sulfur and oxygen, each of which is optionally substituted with one or more of the following groups: C₁₋₃alkyl, halo and cyclopropyl.

In a further embodiment R₁ is selected from the group consisting of: phenyl, a heteroaryl group having 5 or 6 ring atoms in which between one and three of the ring atoms are selected from nitrogen, sulfur and oxygen and a heterocyclyl group having 5 or 6 ring atoms in which one or two of the ring atoms are selected from nitrogen and oxygen, each of which is optionally substituted with one or more of the following groups: C₁₋₃alkyl, halo and cyclopropyl.

In another embodiment R₁ is selected from the group consisting of: phenyl, a heteroaryl group having 5 or 6 ring atoms in which between one and three of the ring atoms are selected from nitrogen, sulfur and oxygen and a heterocyclyl group having 5 or 6 ring atoms in which one or two of the ring atoms are selected from nitrogen and oxygen, each of which is optionally substituted with one or more of the following groups: methyl, chloro, fluoro and cyclopropyl.

In still a further embodiment R₁ is selected from the group consisting of: phenyl, a heteroaryl group having 5 or 6 ring atoms in which between one and three of the ring atoms are selected from nitrogen, sulfur and oxygen and a heterocyclyl group having 6 ring atoms in which one or two of the ring atoms are selected from nitrogen and oxygen, each of which is optionally substituted with one or two of the following groups: methyl, chloro, fluoro and cyclopropyl.

In a further embodiment R₁ is selected from the group consisting of: phenyl, a heteroaryl group having 5 or 6 ring atoms in which between one and three of the ring atoms are selected from nitrogen, sulfur and oxygen and a heterocyclyl group having 6 ring atoms in which one or two of the ring atoms are selected from nitrogen and oxygen, each of which is optionally substituted with one of the following groups: methyl, chloro, fluoro and cyclopropyl.

In yet another embodiment R₁ is selected from the group consisting of:

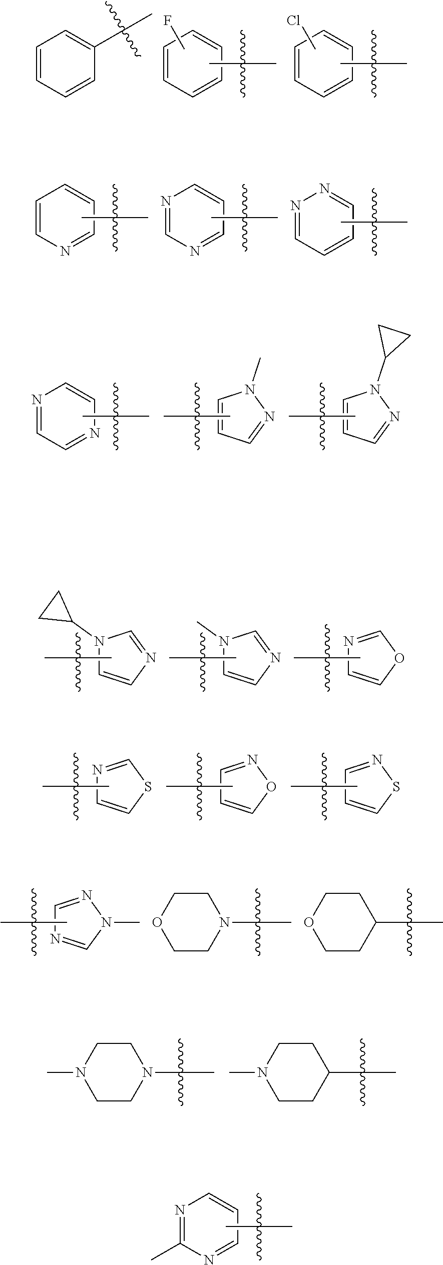

In one embodiment R₂ is selected from the group consisting of:
(i) C₆₋₁₀aryl, a heteroaryl group having between 5 and 10 ring atoms in which one or more of the ring atoms are nitrogen, a heterocyclyl group having between 5 and 10 ring atoms in which one or more of the ring atoms are nitrogen or oxygen, each of which is optionally substituted with one or more of the following groups: NR₅R₆, C₁₋₆alkyl, C₁₋₆alkoxy and halo; and
(ii)-X-Y,
in which X is selected from the group consisting of: a heterocyclyl group having between 5 and 10 ring atoms in which one or more of the ring atoms are nitrogen or oxygen, C₆₋₁₀aryl and a heteroaryl group having between 5 and 10 ring atoms in which one or more of the ring atoms are selected from nitrogen, sulfur and oxygen; and Y is selected from the group consisting of: C₆₋₁₀aryl and a heteroaryl group having between 5 and 10 ring atoms in which one or more of the ring atoms are nitrogen, and wherein the C₆₋₁₀aryl and heteroaryl groups of Y are optionally substituted with one or more of the following groups: NR₅R₆, C₁₋₆alkyl, C₁₋₆alkoxy and halo.

In another embodiment R₂ is selected from the group consisting of:
(i) phenyl, a heteroaryl group having between 5 and 10 ring atoms in which one or more of the ring atoms are nitrogen, a heterocyclyl group having between 5 and 10 ring atoms in which one or more of the ring atoms are nitrogen or oxygen, each of which is optionally substituted with one or more of the following groups: NR₅R₆, C₁₋₆alkyl, C₁₋₆alkoxy and halo; and
(ii)-X-Y,
in which X is selected from the group consisting of: a heterocyclyl group having between 5 and 10 ring atoms in which one or more of the ring atoms are nitrogen, phenyl and a heteroaryl group having between 5 or 6 ring atoms in which one or more of the ring atoms are nitrogen; and Y is selected from the group consisting of: phenyl and a heteroaryl group having 5 or 6 ring atoms in which one or more of the ring atoms are nitrogen, and wherein the phenyl and heteroaryl groups of Y are optionally substituted with one or more of the following groups: NR₅R₆, C₁₋₆alkyl, C₁₋₆alkoxy and halo.

In a further embodiment R₂ is selected from the group consisting of:
(i) a heteroaryl group having between 5 and 10 ring atoms in which one or more of the ring atoms are nitrogen, a heterocyclyl group having between 5 and 10 ring atoms in which one or more of the ring atoms are nitrogen or oxygen, each of which is optionally substituted with one or more of the following groups: NR₅R₆, C₁₋₆alkyl, C₁₋₆alkoxy and halo; and
(ii)-X-Y,
in which X is selected from the group consisting of: a heterocyclyl group having between 5 and 10 ring atoms in which one or more of the ring atoms are nitrogen, phenyl; and Y is selected from the group consisting of: phenyl and a heteroaryl group having 5 or 6 ring atoms in which one or more of the ring atoms are nitrogen, and wherein the phenyl and heteroaryl groups of Y are optionally substituted with one or more of the following groups: NR₅R₆, C₁₋₆alkyl, C₁₋₆alkoxy and halo.

In yet another embodiment R₂ is selected from the group consisting of:
(i) a heterocyclyl group having between 5 and 10 ring atoms in which one or more of the ring atoms are nitrogen or oxygen, each of which is optionally substituted with one or more of the following groups: NR₅R₆, C₁₋₆alkyl, C₁₋₆alkoxy and halo; and
(ii)-X-Y,
in which X is selected from the group consisting of: a heterocyclyl group having 5 or 6 ring atoms in which one or more of the ring atoms are nitrogen; and Y is selected from the group consisting of: phenyl and a heteroaryl group having 5 or 6 ring atoms in which one or more of the ring atoms are nitrogen, and wherein the phenyl and heteroaryl groups of Y are optionally substituted with one or more of the following groups: NR₅R₆, C₁₋₆alkyl, C₁₋₆alkoxy and halo.

In still a further embodiment R₂ is selected from the group consisting of:
(i) a heterocyclyl group having between 5 and 10 ring atoms in which between one and four of the ring atoms are nitrogen or oxygen, each of which is optionally substituted with one or more of the following groups: NR₅R₆, C₁₋₆alkyl, C₁₋₆alkoxy and halo; and
(ii)-X-Y,
in which X is selected from the group consisting of: a heterocyclyl group having 5 or 6 ring atoms in which one or more of the ring atoms are nitrogen; and Y is selected from the group consisting of: phenyl and a heteroaryl group having 5 or 6 ring atoms in which one or more of the ring atoms are nitrogen, and wherein the phenyl and heteroaryl groups of Y are optionally substituted with one or more of the following groups: NH₂, N(Me)₂, C₁₋₆alkyl, methoxy, ethoxy and halo.

In another embodiment R₂ is selected from the group consisting of:
(i) a heterocyclyl group having between 5 and 10 ring atoms in which between one and four of the ring atoms are nitrogen or oxygen, each of which is optionally substituted with one or more of the following groups: NH₂, NMe₂, C₁₋₆alkyl, methoxy, ethoxy and halo; and
(ii)-X-Y,
in which X is selected from the group consisting of: a heterocyclyl group having 5 or 6 ring atoms in which one or more of the ring atoms are nitrogen; and Y is selected from the group consisting of: phenyl and a heteroaryl group having 5 or 6 ring atoms in which one or more of the ring atoms are nitrogen, and wherein the phenyl and heteroaryl groups of Y are optionally substituted with one or more of the following groups: C₁₋₆alkyl, methoxy, ethoxy and halo.

In yet another embodiment R₂ is selected from the group consisting of:
(i) a heterocyclyl group having between 5 and 10 ring atoms in which between one and four of the ring atoms are nitrogen or oxygen, each of which is optionally substituted with one or more of the following groups: NH₂, N(Me)₂, methyl, methoxy and halo; and
(ii)-X-Y,
in which X is selected from the group consisting of: a heterocyclyl group having 5 or 6 ring atoms in which one or more of the ring atoms are nitrogen; and Y is selected from the group consisting of: phenyl and a heteroaryl group having 5 or 6 ring atoms in which one or more of the ring atoms are nitrogen, and wherein the phenyl and heteroaryl groups of Y are optionally substituted with between one and three of the following groups: methyl, methoxy and halo.

In still a further embodiment R₂ is selected from the group consisting of:
(i) a heterocyclyl group having between 5 and 10 ring atoms in which between one and four of the ring atoms are nitrogen or oxygen, each of which is optionally substituted with between one and three of the following groups: NH₂, N(Me)₂, methyl and methoxy, and
(ii)-X-Y,
in which X is selected from the group consisting of: a heterocyclyl group having 5 or 6 ring atoms in which one or two of the ring atoms are nitrogen; and Y is selected from the group consisting of: phenyl and a heteroaryl group having 5 or 6 ring atoms in which between one and three of the ring atoms are nitrogen, and wherein the phenyl and heteroaryl groups of Y are optionally substituted with one or two of the following groups: methyl, methoxy, fluoro and chloro.

In still a further embodiment R₂ is selected from the group consisting of:
(i) a heterocyclyl group having between 5 and 10 ring atoms in which between one and four of the ring atoms are nitrogen or oxygen, each of which is optionally substituted with between one and three of the following groups: NH₂, N(Me)₂, methyl and methoxy, and
(ii)-X-Y,
in which X is selected from the group consisting of: a heterocyclyl group having 6 ring atoms in which one or two of the ring atoms are nitrogen; and Y is selected from the group consisting of: phenyl and a heteroaryl group having 5 or 6 ring atoms in which one or two of the ring atoms are nitrogen, and wherein the phenyl and heteroaryl groups of Y are optionally substituted with one or two of the following groups: methyl, methoxy, fluoro and chloro.

In yet another embodiment R₂ may be selected from the group consisting of:

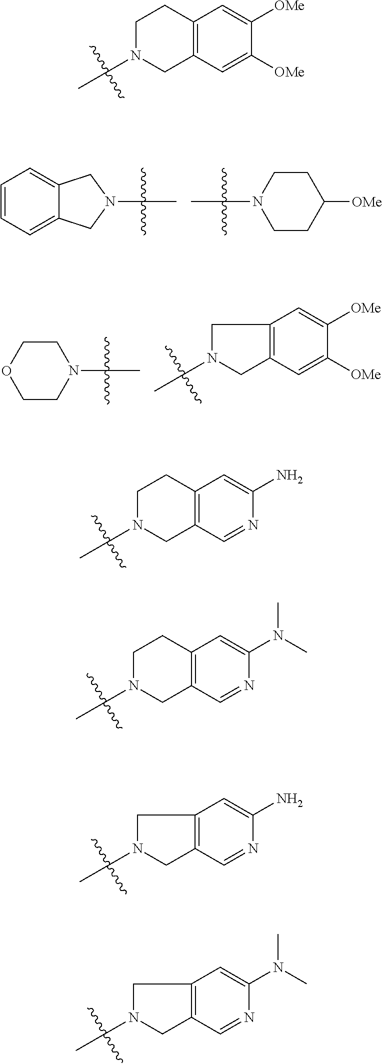

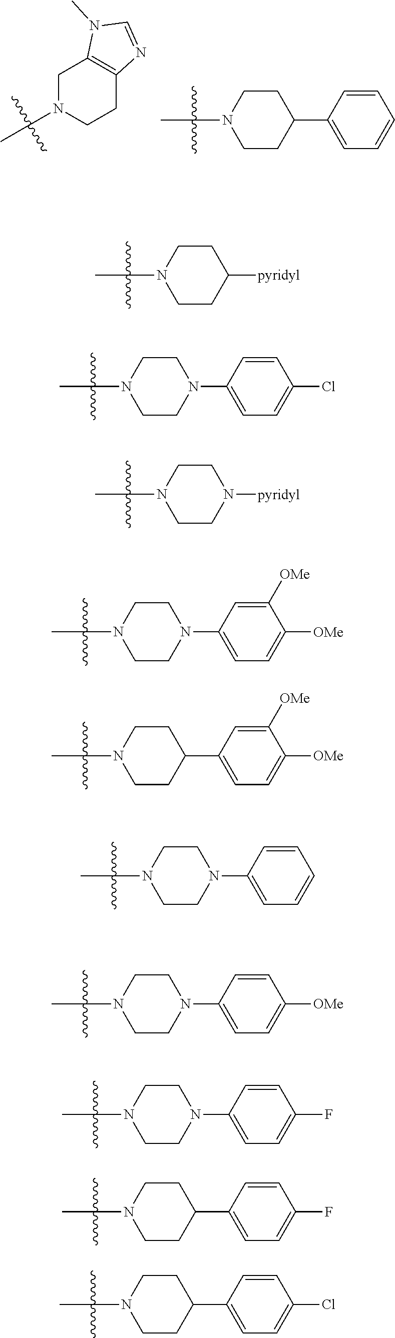

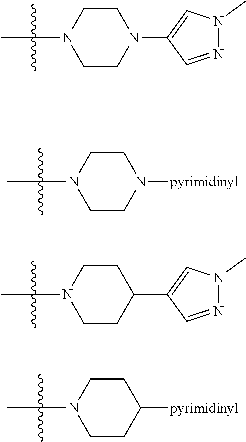

In one embodiment R₃ is selected from the group consisting of: halo, C₁₋₆ alkoxy, OH, CN, —OC(O)R₄, —C(O)R₄, —C(O)NR₅R₆, —C(O)OR₄, NR₅R₆, C₁₋₁₀alkyl, C₂₋₁₀alkenyl, C₂₋₁₀alkynyl; C₃₋₁₀cycloalkyl, C₆₋₁₀aryl and a heteroaryl group having between 5 and 10 ring atoms in which one or more of the ring atoms are selected from nitrogen, sulfur and oxygen.

In another embodiment R₃ is selected from the group consisting of: halo, C₁₋₆ alkoxy, OH, CN, —C(O)R₄, —C(O)NR₅R₆, —C(O)OR₄, NR₅R₆, C₁₋₆alkyl, C₂₋₆alkenyl, C₂₋₆alkynyl; C₃₋₁₀cycloalkyl, C₆₋₁₀aryl and a heteroaryl group having between 5 or 6 ring atoms in which one or more of the ring atoms are selected from nitrogen, sulfur and oxygen.

In a further embodiment R₃ is selected from the group consisting of: halo, C₁₋₆ alkoxy, OH, CN, NR₅R₆, C₁₋₆alkyl, C₂₋₆alkenyl, C₂₋₆alkynyl, C₃₋₆cycloalkyl, C₆₋₁₀aryl and a heteroaryl group having between 5 or 6 ring atoms in which one or more of the ring atoms are selected from nitrogen, sulfur and oxygen.

In yet another embodiment R₃ is selected from the group consisting of: halo, C₁₋₄ alkoxy, OH, CN, NR₅R₆, C₁₋₄alkyl, C₃₋₆cycloalkyl, C₆₋₁₀aryl and a heteroaryl group having between 5 or 6 ring atoms in which one or more of the ring atoms are selected from nitrogen, sulfur and oxygen.

In still a further embodiment R₃ is selected from the group consisting of: halo, C₁₋₄ alkoxy, OH, CN, NR₅R₆, C₁₋₄alkyl, C₃₋₆cycloalkyl, phenyl and a heteroaryl group having between 5 or 6 ring atoms in which one or more of the ring atoms are selected from nitrogen, sulfur and oxygen.

In yet another embodiment R₃ is selected from the group consisting of: halo, C₁₋₄ alkoxy, OH, CN, C₁₋₄alkyl, phenyl and a heteroaryl group having between 5 or 6 ring atoms in which one or more of the ring atoms are selected from nitrogen, sulfur and oxygen.

In a further embodiment R₃ is selected from the group consisting of: halo, C₁₋₄ alkoxy, OH, CN, C₁₋₄alkyl and phenyl.

In still a further embodiment R₃ is selected from the group consisting of: halo, C₁₋₄ alkoxy, CN and C₁₋₄alkyl.

In yet another embodiment R₃ is selected from the group consisting of: halo, C₁₋₄ alkoxy, CN, C₁₋₄alkyl and phenyl.

In still a further embodiment R₃ is selected from the group consisting of: chloro, fluoro, methoxy, ethoxy and CN.

In another embodiment R₃ is selected from the group consisting of: fluoro, methoxy, ethoxy and CN.

In another embodiment R₃ is selected from the group consisting of: fluoro, methoxy and CN.

In one embodiment R₄ is H or CH₃.

In one embodiment R₅ and R₆ are independently selected from H and CH₃.

In one embodiment n is 1.

In one embodiment m is 0, 1 or 2. In another embodiment m is 0 or 1.

In another aspect there is provided a compound of formula (II), or a pharmaceutically acceptable salt or prodrug thereof:

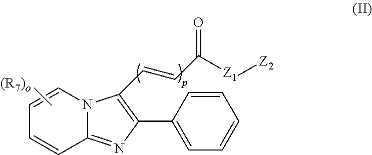

wherein:
R₇ is selected from the group consisting of: halo, OC₁₋₆ alkyl, OH, CN, NO₂, —OC(O)R₄, —C(O)R₄, —C(O)NR₅R₆, —C(O)OR₄, —OS(O)₂R₄, NR₅R₆, SR₄, C₁₋₁₀alkyl, C₂₋₁₀alkenyl, C₂₋₁₀alkynyl; C₃₋₁₀cycloalkyl, C₆₋₁₀aryl and a heteroaryl group having between 5 and 10 ring atoms in which one or more of the ring atoms are selected from nitrogen, sulfur and oxygen;
Z₁ is a heterocyclyl group having between 5 and 10 ring atoms in which one or more of the ring atoms are selected from nitrogen, sulfur and oxygen;
Z₂ is selected from: phenyl, a heteroaryl group having between 5 and 10 ring atoms in which one or more of the ring atoms are selected from nitrogen, sulfur and oxygen, and a heterocyclyl group having between 5 and 10 ring atoms in which one or more of the ring atoms are selected from nitrogen, sulfur and oxygen, each of which is optionally substituted with one or more of the following groups: NR₅R₆, C₁₋₆alkyl, OC₁₋₆alkyl and halo;
p is 1 or 2; and,
o is an integer between 0 and 4,
and wherein R₄ to R₆ are as defined in the first aspect.

In some embodiments R₇ is selected from the group consisting of: halo, methoxy, OH, CN, NR₅R₆ and methyl.

In one embodiment Z₁ is a heterocyclyl group having 5 or 6 ring atoms in which one or more of the ring atoms are nitrogen, oxygen or sulfur.

In another embodiment Z₁ is a heterocyclyl group having 5 or 6 ring atoms in which one or two of the ring atoms are nitrogen, oxygen or sulfur.

In a further embodiment Z₁ is a heterocyclyl group having 5 or 6 ring atoms in which one or two of the ring atoms are nitrogen.

In a further embodiment Z₁ is a heterocyclyl group having 6 ring atoms in which one or two of the ring atoms are nitrogen.

In yet another embodiment Z₁ is:

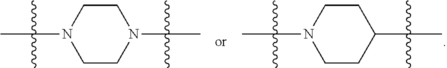 [SMILES: *N1CCN(*)CC1 ; *C1CCN(*)CC1]

In a further embodiment Z₂ is selected from the group consisting of: phenyl and a heteroaryl group having 5 or 6 ring atoms in which between one and three of the ring atoms are nitrogen, and wherein the phenyl and heteroaryl groups are optionally substituted with between one and three of the following groups: NH₂, N(Me)₂, methyl, methoxy and halo.

In yet another embodiment Z₂ is selected from the group consisting of: phenyl and a heteroaryl group having 5 or 6 ring atoms in which between one and three of the ring atoms are nitrogen, and wherein the phenyl and heteroaryl groups are optionally substituted with one or two of the following groups: NH₂, N(Me)₂, methyl, methoxy, fluoro and chloro.

In still a further embodiment Z₂ is selected from the group consisting of: phenyl and a heteroaryl group having 5 or 6 ring atoms in which one or two of the ring atoms are nitrogen, and wherein the phenyl and heteroaryl groups are optionally substituted with one or two of the following groups: NH₂, N(Me)₂, methyl, methoxy, fluoro and chloro.

In one embodiment o is 0 or 1. In another embodiment o is 0.

In one embodiment p is 1.

Exemplary compounds according to formula (I) and (II) include:

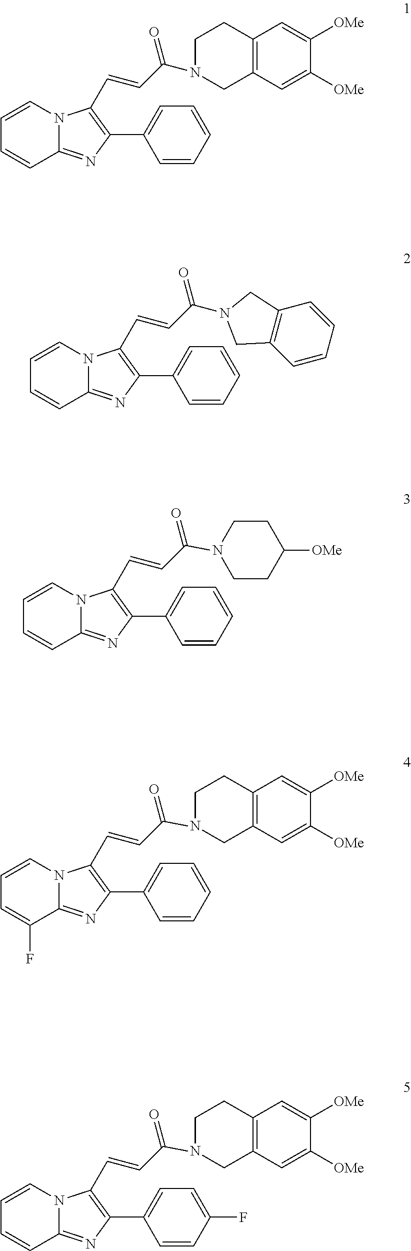 [SMILES: COc1cc2c(cc1OC)CN(C(=O)/C=C/c1c(-c3ccccc3)nc3ccccn13)CC2 ; O=C(/C=C/c1c(-c2ccccc2)nc2ccccn12)N1Cc2ccccc2C1 ; COC1CCN(C(=O)/C=C/c2c(-c3ccccc3)nc3ccccn23)CC1 ; COc1cc2c(cc1OC)CN(C(=O)/C=C/c1c(-c3ccccc3)nc3c(F)cccn13)CC2 ; COc1cc2c(cc1OC)CN(C(=O)/C=C/c1c(-c3ccc(F)cc3)nc3ccccn13)CC2]

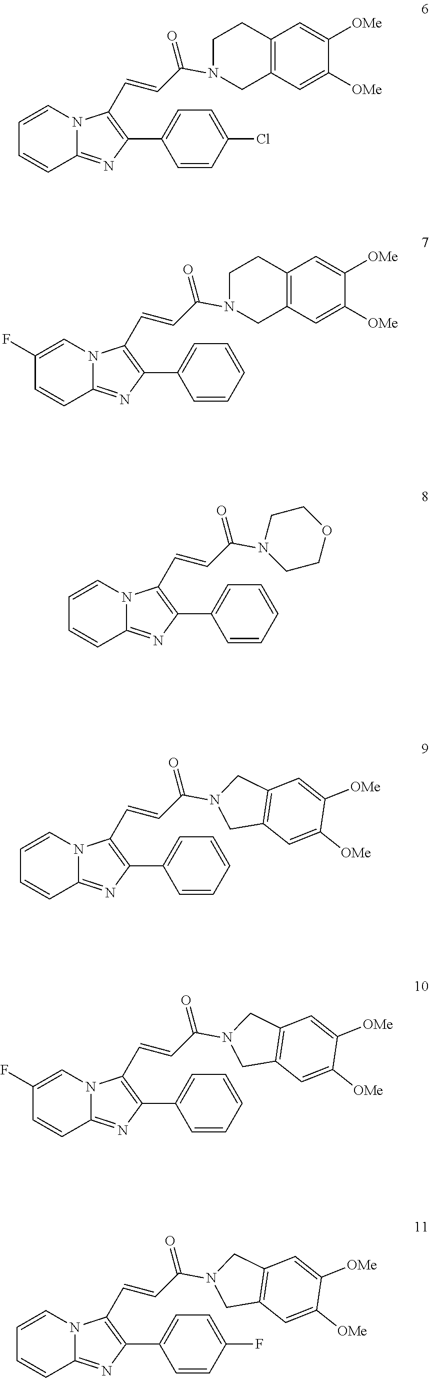 [SMILES: COc1cc2c(cc1OC)CN(C(=O)/C=C/c1c(-c3ccc(Cl)cc3)nc3ccccn13)CC2 ; COc1cc2c(cc1OC)CN(C(=O)/C=C/c1c(-c3ccccc3)nc3ccc(F)cn13)CC2 ; O=C(/C=C/c1c(-c2ccccc2)nc2ccccn12)N1CCOCC1 ; COc1cc2c(cc1OC)CN(C(=O)/C=C/c1c(-c3ccccc3)nc3ccccn13)C2 ; COc1cc2c(cc1OC)CN(C(=O)/C=C/c1c(-c3ccccc3)nc3ccc(F)cn13)C2 ; COc1cc2c(cc1OC)CN(C(=O)/C=C/c1c(-c3ccc(F)cc3)nc3ccccn13)C2]

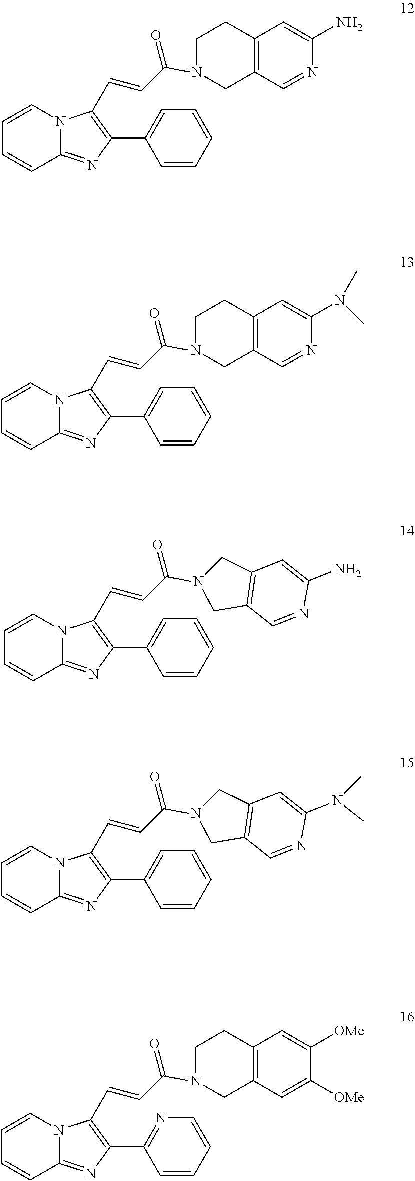 [SMILES: Nc1cc2c(cn1)CN(C(=O)/C=C/c1c(-c3ccccc3)nc3ccccn13)CC2 ; CN(C)c1cc2c(cn1)CN(C(=O)/C=C/c1c(-c3ccccc3)nc3ccccn13)CC2 ; Nc1cc2c(cn1)CN(C(=O)/C=C/c1c(-c3ccccc3)nc3ccccn13)C2 ; CN(C)c1cc2c(cn1)CN(C(=O)/C=C/c1c(-c3ccccc3)nc3ccccn13)C2 ; COc1cc2c(cc1OC)CN(C(=O)/C=C/c1c(-c3ccccn3)nc3ccccn13)CC2]

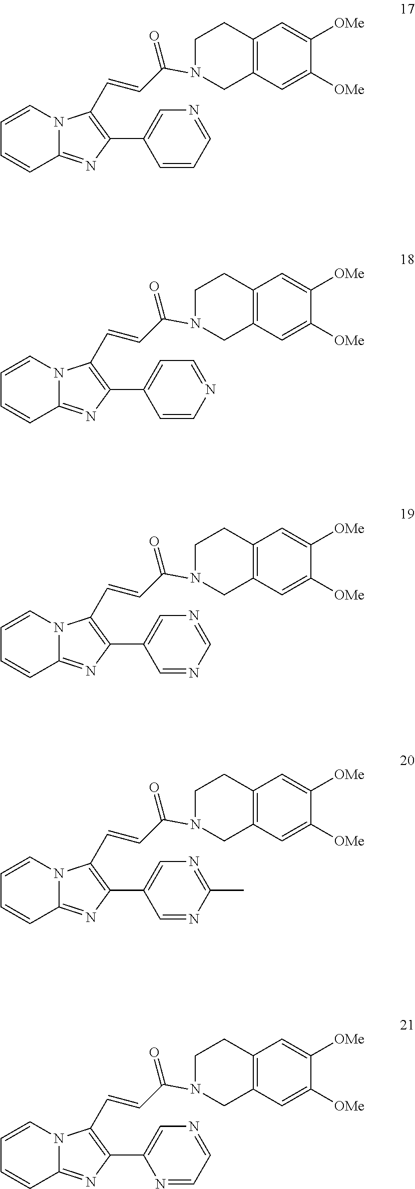 [SMILES: COc1cc2c(cc1OC)CN(C(=O)/C=C/c1c(-c3cccnc3)nc3ccccn13)CC2 ; COc1cc2c(cc1OC)CN(C(=O)/C=C/c1c(-c3ccncc3)nc3ccccn13)CC2 ; COc1cc2c(cc1OC)CN(C(=O)/C=C/c1c(-c3cncnc3)nc3ccccn13)CC2 ; COc1cc2c(cc1OC)CN(C(=O)/C=C/c1c(-c3cnc(C)nc3)nc3ccccn13)CC2 ; COc1cc2c(cc1OC)CN(C(=O)/C=C/c1c(-c3cnccn3)nc3ccccn13)CC2]

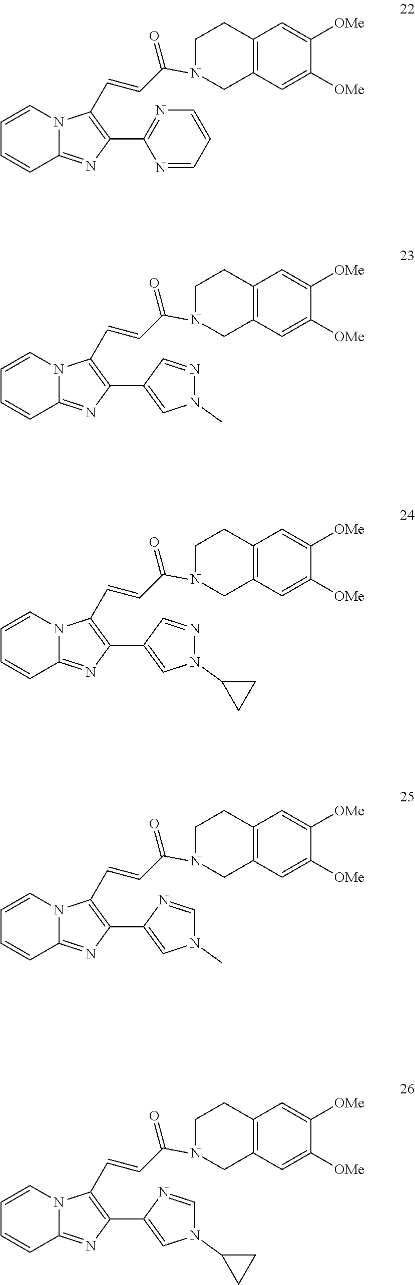 [SMILES: COc1cc2c(cc1OC)CN(C(=O)/C=C/c1c(-c3ncccn3)nc3ccccn13)CC2 ; COc1cc2c(cc1OC)CN(C(=O)/C=C/c1c(-c3cnn(C)c3)nc3ccccn13)CC2 ; COc1cc2c(cc1OC)CN(C(=O)/C=C/c1c(-c3cnn(C4CC4)c3)nc3ccccn13)CC2 ; COc1cc2c(cc1OC)CN(C(=O)/C=C/c1c(-c3cn(C)cn3)nc3ccccn13)CC2 ; COc1cc2c(cc1OC)CN(C(=O)/C=C/c1c(-c3cn(C4CC4)cn3)nc3ccccn13)CC2]

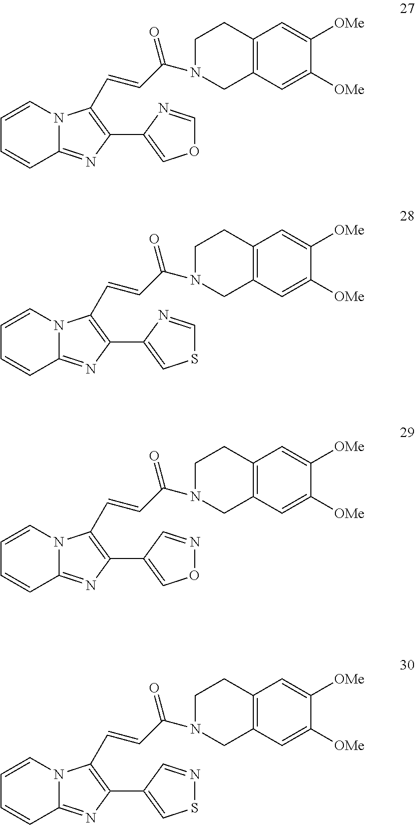 [SMILES: COc1cc2c(cc1OC)CN(C(=O)/C=C/c1c(-c3cocn3)nc3ccccn13)CC2 ; COc1cc2c(cc1OC)CN(C(=O)/C=C/c1c(-c3cscn3)nc3ccccn13)CC2 ; COc1cc2c(cc1OC)CN(C(=O)/C=C/c1c(-c3cnoc3)nc3ccccn13)CC2 ; COc1cc2c(cc1OC)CN(C(=O)/C=C/c1c(-c3cnsc3)nc3ccccn13)CC2]

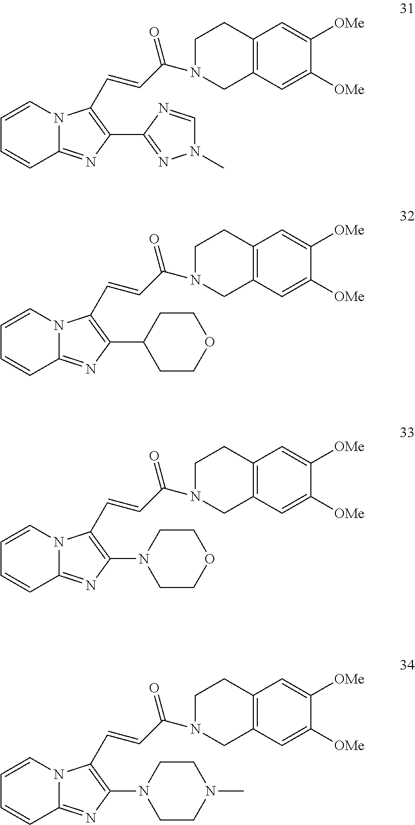 [SMILES: COc1cc2c(cc1OC)CN(C(=O)/C=C/c1c(-c3ncn(C)n3)nc3ccccn13)CC2 ; COc1cc2c(cc1OC)CN(C(=O)/C=C/c1c(C3CCOCC3)nc3ccccn13)CC2 ; COc1cc2c(cc1OC)CN(C(=O)/C=C/c1c(N3CCOCC3)nc3ccccn13)CC2 ; COc1cc2c(cc1OC)CN(C(=O)/C=C/c1c(N3CCN(C)CC3)nc3ccccn13)CC2]

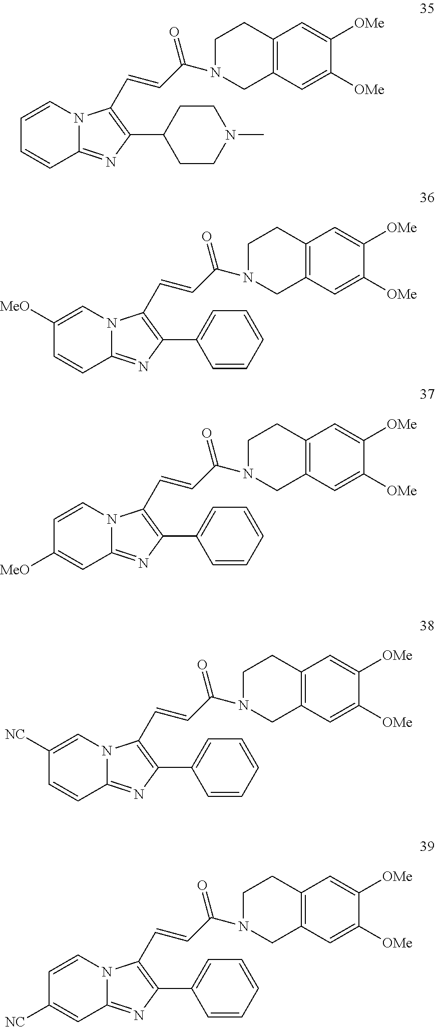 [SMILES: COc1cc2c(cc1OC)CN(C(=O)/C=C/c1c(C3CCN(C)CC3)nc3ccccn13)CC2 ; COc1ccc2nc(-c3ccccc3)c(/C=C/C(=O)N3CCc4cc(OC)c(OC)cc4C3)n2c1 ; COc1ccn2c(/C=C/C(=O)N3CCc4cc(OC)c(OC)cc4C3)c(-c3ccccc3)nc2c1 ; COc1cc2c(cc1OC)CN(C(=O)/C=C/c1c(-c3ccccc3)nc3ccc(C#N)cn13)CC2 ; COc1cc2c(cc1OC)CN(C(=O)/C=C/c1c(-c3ccccc3)nc3cc(C#N)ccn13)CC2]

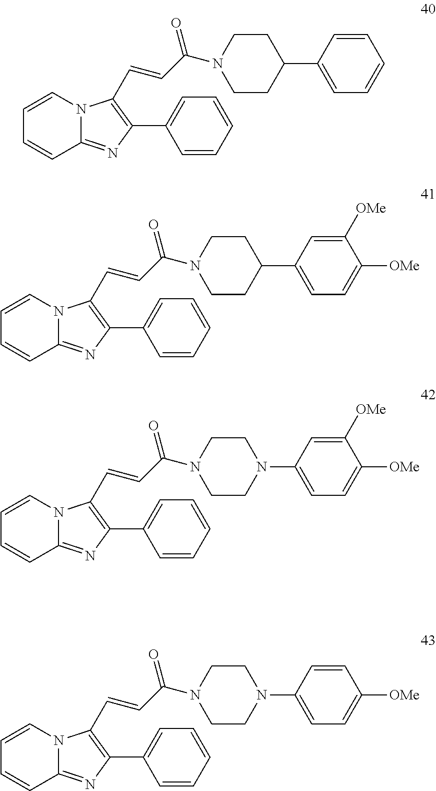 [SMILES: O=C(/C=C/c1c(-c2ccccc2)nc2ccccn12)N1CCC(c2ccccc2)CC1 ; COc1ccc(C2CCN(C(=O)/C=C/c3c(-c4ccccc4)nc4ccccn34)CC2)cc1OC ; COc1ccc(N2CCN(C(=O)/C=C/c3c(-c4ccccc4)nc4ccccn34)CC2)cc1OC ; COc1ccc(N2CCN(C(=O)/C=C/c3c(-c4ccccc4)nc4ccccn34)CC2)cc1]

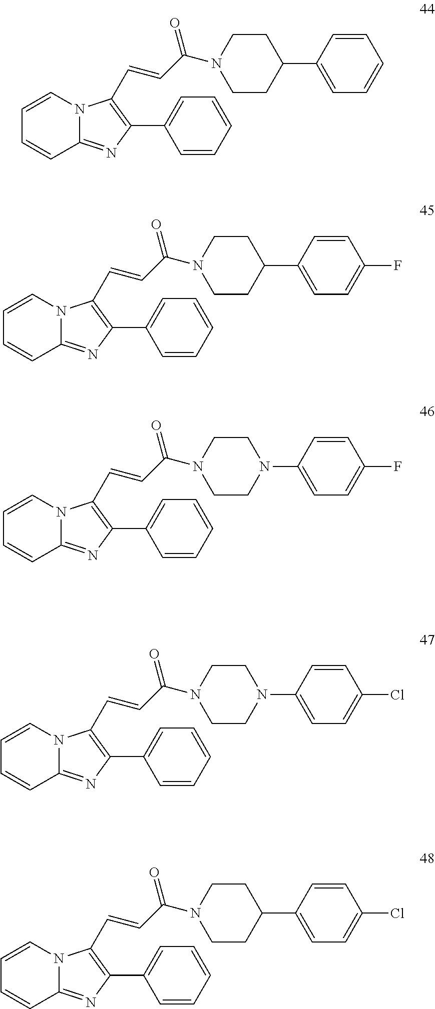 [SMILES: O=C(/C=C/c1c(-c2ccccc2)nc2ccccn12)N1CCC(c2ccccc2)CC1 ; O=C(/C=C/c1c(-c2ccccc2)nc2ccccn12)N1CCC(c2ccc(F)cc2)CC1 ; O=C(/C=C/c1c(-c2ccccc2)nc2ccccn12)N1CCN(c2ccc(F)cc2)CC1 ; O=C(/C=C/c1c(-c2ccccc2)nc2ccccn12)N1CCN(c2ccc(Cl)cc2)CC1 ; O=C(/C=C/c1c(-c2ccccc2)nc2ccccn12)N1CCC(c2ccc(Cl)cc2)CC1]

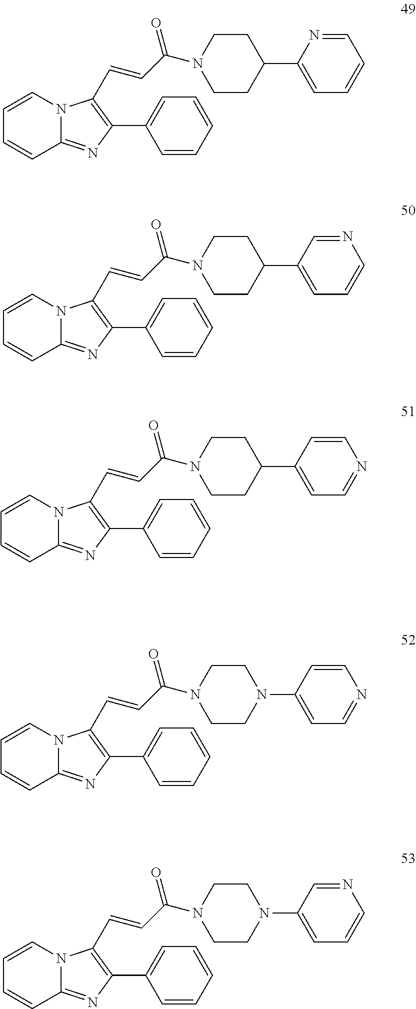 [SMILES: O=C(/C=C/c1c(-c2ccccc2)nc2ccccn12)N1CCC(c2ccccn2)CC1 ; O=C(/C=C/c1c(-c2ccccc2)nc2ccccn12)N1CCC(c2cccnc2)CC1 ; O=C(/C=C/c1c(-c2ccccc2)nc2ccccn12)N1CCC(c2ccncc2)CC1 ; O=C(/C=C/c1c(-c2ccccc2)nc2ccccn12)N1CCN(c2ccncc2)CC1 ; O=C(/C=C/c1c(-c2ccccc2)nc2ccccn12)N1CCN(c2cccnc2)CC1]

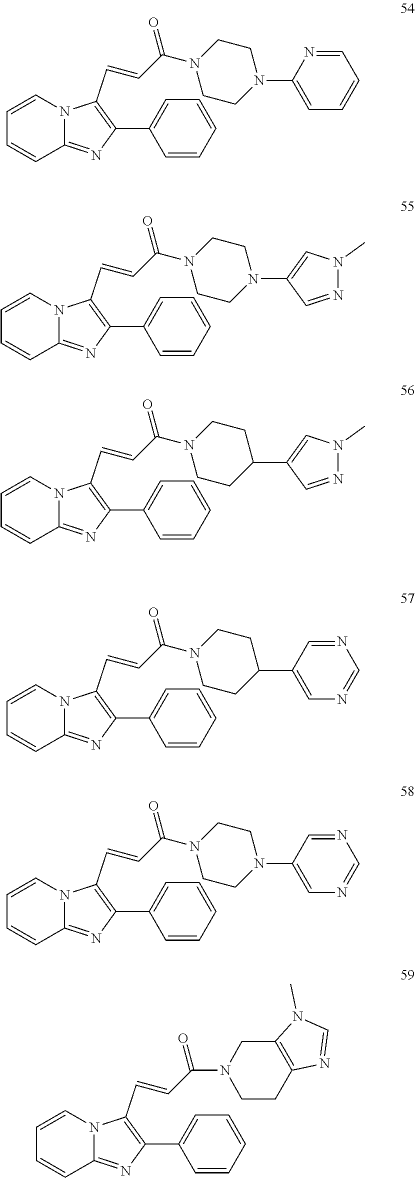 [SMILES: O=C(/C=C/c1c(-c2ccccc2)nc2ccccn12)N1CCN(c2ccccn2)CC1 ; Cn1cc(N2CCN(C(=O)/C=C/c3c(-c4ccccc4)nc4ccccn34)CC2)cn1 ; Cn1cc(C2CCN(C(=O)/C=C/c3c(-c4ccccc4)nc4ccccn34)CC2)cn1 ; O=C(/C=C/c1c(-c2ccccc2)nc2ccccn12)N1CCC(c2cncnc2)CC1 ; O=C(/C=C/c1c(-c2ccccc2)nc2ccccn12)N1CCN(c2cncnc2)CC1 ; Cn1cnc2c1CN(C(=O)/C=C/c1c(-c3ccccc3)nc3ccccn13)CC2]

In one embodiment the compound of formula (I) is selected from any one or more of the above compounds 1 to 59, in any combination.

In another embodiment the compound of formula (II) is selected from any one or more of the above compounds 40 to 58, in any combination.

Compounds of the formula (II) may also be used in any of the first to fourth and seventh to tenth aspects.

Compounds of formulae (I) and (II) include a carbon-carbon double bond which links the carbonyl group to the imidazo[1,2-a]pyridine ring. The present disclosure extends to all geometric isomers, i.e. to both cis and trans isomers. Compounds of formulae (I) and (II) may exist as tautomeric isomers. The present disclosure extends to all tautomers.

Compounds of the formulae (I) and (II) are also taken to include hydrates and solvates. Solvates are complexes formed by association of molecules of a solvent with a compound of the formulae (I) or (II). In the case of compounds of the formulae (I) and (II) that are solids, it will be understood by those skilled in the art that such compounds may exist in different crystalline or polymorphic forms, all of which are intended to be within the scope of the present disclosure.

The compounds of formulae (I) and (II) may be in the form of pharmaceutically acceptable salts. Such salts are well known to those skilled in the art. S. M. Berge et al. describe pharmaceutically acceptable salts in detail in J. Pharmaceutical Sciences, 1977, 66: 1-19. Pharmaceutically acceptable salts can be prepared in situ during the final isolation and purification of compounds of the formulae (I) and (II), or separately by reacting the free base compound with a suitable organic acid. Suitable pharmaceutically acceptable acid addition salts of the compounds of the present disclosure may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycioaliphatic, aromatic, heterocyclic carboxylic and sulfonic classes of organic acids, examples of which are formic, acetic, propionic, succinic, giycolic, gluconic, lactic, malic, tartaric, citric, ascorbic, glucoronic, fumaric, maleic, pyruvic, alkyl sulfonic, arylsulfonic, aspartic, glutamic, benzoic, anthranilic, mesylic, salicylic, p-hydroxybenzoic, phenylacetic, pamoic, pantothenic, sulfanilic, cyclohexylaminosulfonic, stearic, algenic, β-hydroxybutyric and galacturonic acids. Suitable pharmaceutically acceptable base addition salts of the compounds of the present disclosure include metallic salts made from lithium, sodium, potassium, magnesium, calcium, aluminium and zinc, and organic salts made from organic bases such as choline, diethanolamine and morpholine. Alternatively, organic salts made from N,N′-dibenzylethylenediamine, chloroprocaine, choline, diethanolamine, ethylenediamine, meglumine (N-methylglucamine), procaine, ammonium salts, quaternary salts, such as tetramethylammonium salt, amino acid addition salts, such as salts with glycine and arginine.

The compounds of formulae (I) and (II) also extend to include all derivatives, such as prodrugs, with physiologically cleavable leaving groups that can be cleaved in vivo to provide the compounds of formulae (I) and (II).

Compounds of the formula (I) may be synthesized as described below in Scheme 1.

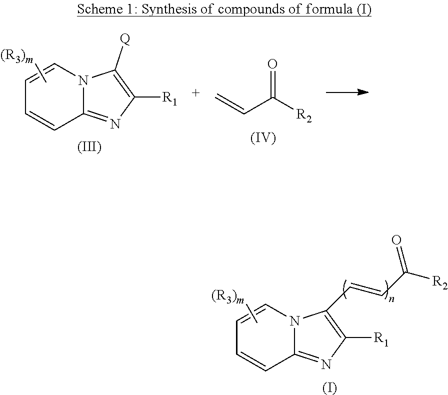

Imidazo[1,2-a]pyridine derivative (III), in which Q is a leaving group such as for example a halide, may be reacted with α,β-unsaturated ketone (IV) to afford compounds of the formula (I). Armed with this synthetic procedure and the common general knowledge, those skilled in the art will readily be able to prepare all compounds embraced by formulae (I) and (II).

The compounds of formulae (I) and (II) find use in the treatment of fibrosis. Fibrosis can occur in many tissues within the body, typically as a result of inflammation or damage, and the present disclosure contemplates treatment of any and all kinds of fibrosis occurring in any part of the body. In some embodiments the fibrosis is in the lungs, for example pulmonary fibrosis (including idiopathic pulmonary fibrosis), cystic fibrosis or fibrosis resulting from radiation, such as for example radiotherapy. In other embodiments the fibrosis may be renal fibrosis, cardiac fibrosis, liver fibrosis, such as for example cirrhosis or bridging fibrosis. In alternative embodiments the fibrosis may be arthrofibrosis, nephrogenic systemic fibrosis, retroperitoneal fibrosis, scleroderma/systemic sclerosis, myelofibrosis or mediastinal fibrosis. In other embodiments the fibrosis may be associated with arterial stiffness, Crohn's disease, Dupuytren's contracture, keloids, Peyronie's disease or adhesive capsulitis.

The compounds of formulae (I) and (II) also find use in the treatment of fibrosis that is associated with a coronavirus. Coronaviruses are a group of related RNA viruses that cause diseases and various complications in mammals and birds. In some embodiments the coronavirus is a MERS-coronavirus, SARS-coronavirus or SARS-coronavirus-2 (commonly referred to as “Covid-19”). Reports have emerged of pulmonary fibrotic disease occurring regularly in subjects diagnosed with Covid-19, ranging from fibrosis associated with pneumonia to severe acute lung injury, in which there is widespread fibrotic change. As such, in some embodiments, the fibrosis is pulmonary fibrosis that is caused by, associated with, or otherwise related to a coronavirus infection in the subject. In this context the term “associated with, or otherwise related to” means that the pulmonary fibrosis, or a symptom thereof, has at least some connection whether direct or indirect, to the coronavirus infection in the subject.

In subjects who have recovered from Covid-19, progressive, fibrotic irreversible interstitial lung disease (which is characterised by declining lung function, increasing extent of fibrosis on CT, worsening symptoms and quality of life, and early mortality) may arise with varying degrees of frequency. In this regard, removal of the cause of lung damage does not preclude the development of progressive, fibrotic irreversible interstitial lung disease. In addition, even a relatively small degree of residual, but non-progressive fibrosis could result in considerable morbidity and mortality in an older population of patients who have had COVID-19, many of whom will also have pre-existing pulmonary conditions. Accordingly, in some embodiments, the subject may be suffering from a coronavirus infection, or alternatively the subject may have recovered from a coronavirus infection but may still be suffering from the effects of pulmonary fibrosis or fibrotic damage or symptoms thereof, despite no longer having the virus. In treating fibrosis, the compounds of formulae (I) or (II) may serve to reduce fibrotic damage in the subject.

In some embodiments the compounds of formulae (I) or (II) may be administered together with one or more anti-inflammatory compounds. Anti-inflammatory compounds will be well known to those skilled in the art. Suitable anti-inflammatory compounds include both steroidal and non-steriodal. Suitable non-steroidal anti-inflammatory compounds include, but are not limited to indomethacin, diclofenac, aceclofenac, sulindac, phenylbutazone, valdecoxib, rofecoxib, celecoxib, tenoxicam, piroxicam, ketoprofen, naproxen, ibuprofen, flurbiprofen and nabumetone. Suitable steriodal anti-inflammatory compounds include, but are not limited to hydrocortisone, cortisone, tixocortal pivalate, prednisolone, methylprednisolone, prednisone, budesonide, desonide, amcinonide, fluocinolone acetonide, fluocinocide, halcinonide, triamcinolone acetonide, beclometasone, dexamethasone, fluocortolone, halometasone and mometasone.

In some embodiments the compounds of formulae (I) or (II) may be administered together with one or more antiviral compounds. Antiviral compounds will be well known to those skilled in the art. Suitable antiviral compounds include, but are not limited to remdesivir, acyclovir, boceprevir, darunavir, entecavir, nelfinavir, penciclovir, pleconaril, rilpivirine, simeprevir and the like.

In addition to treatment, the compounds of formulae (I) and (II) may be used in the prevention or minimisation of fibrosis, lung injury and/or damage in a subject infected with a coronavirus, or in a subject at risk of becoming infected with a coronavirus. Such use may substantially reduce the burden of pulmonary fibrosis by preventing its onset, or otherwise minimising its deleterious effects on the lungs. In some embodiments, the compound of formulae (I) or (II) is administered within a week of the subject being diagnosed with the coronavirus. In other embodiments the compound of formulae (I) or (II) may be administered within a week of onset of ARDS in the subject. Administration within a week of onset of ARDS is likely to optimise effectiveness. In preventing or minimising fibrosis, the compounds of formulae (I) or (II) may serve to prevent or minimise fibrotic damage in the subject.

By virtue of their anti-fibrotic effects, compounds of the formulae (I) and (II) may also find use in minimising the severity of a coronavirus infection by treating, preventing or otherwise lessening the severity of lung injury or damage.

Those skilled in the art will recognise that compounds and pharmaceutical compositions of the disclosure may be administered via any route which delivers an effective amount of the compounds to the tissue or site to be treated. In general, the compounds and compositions may be administered by the parenteral (for example intravenous, intraspinal, subcutaneous or intramuscular), oral, rectal, inhalation or topical route. Administration may be systemic, regional or local.

The particular route of administration to be used in any given circumstance will depend on a number of factors, including the nature of the fibrotic disorder to be treated, the severity and extent of the fibrotic disorder, the required dosage of the particular compound to be delivered and the potential side-effects of the compound.

In general, suitable compositions may be prepared according to methods that are known to those of ordinary skill in the art and may include pharmaceutically acceptable carriers, diluents and/or excipients. The carriers, diluents and excipients must be “acceptable” in terms of being compatible with the other ingredients of the composition, and not deleterious to the recipient thereof.

Examples of pharmaceutically acceptable carriers or diluents are demineralised or distilled water; saline solution; vegetable-based oils such as peanut oil, safflower oil, olive oil, cottonseed oil, maize oil or coconut oil; silicone oils, including polysiloxanes, such as methyl polysiloxane, phenyl polysiloxane and methylphenyl polysiloxane: volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; Cremophor®; cyclodextrins; lower alcohols, for example ethanol or i-propanol; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrrolidone; agar; carrageenan; gum tragacanth or gum acacia and petroleum jelly. Typically, the carrier or carriers will form from about 10% to about 99.9% by weight of the compositions.

Pharmaceutical compositions may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, in a form suitable for parenteral administration, that is, subcutaneous, intramuscular or intravenous injection.

For administration as an injectable solution or suspension, non-toxic parenterally acceptable diluents or carriers can include cyclodextrins (for example Captisol®) Cremophor®, Ringer's solution, isotonic saline, phosphate buffered saline, ethanol and 1,2 propylene glycol. To aid injection and delivery, the compounds may also be added to PEG and non-PEGylated liposomes or micelles with specific targeting tags attached to PEG moieties, such as the RGD peptide or glutathione, for aiding passage across the blood brain barrier.

Some examples of suitable carriers, diluents, excipients and adjuvants for oral use include cyclodextrins, Cremophor®, peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition, these oral formulations may contain suitable flavouring and colourings agents. When used in capsule form, the capsules may be coated with compounds such as glyceryl monostearate or glyceryl distearate that delay disintegration.

Adjuvants typically include emollients, emulsifiers, thickening agents, preservatives, bactericides and buffering agents.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose, or polyethylene glycol. Suitable sweeteners include sucrose, lactose, glucose, aspartame or saccharin. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms suitable for oral administration may contain, in addition to the above agents, a liquid carrier. Suitable liquid carriers include water, oils such as olive oil, peanut oil, sesame oil, sunflower oil, safflower oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethylcellulose, polyvinylpyrrolidone, sodium alginate or cetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

Emulsions for oral administration may further comprise one or more emulsifying agents. Suitable emulsifying agents include dispersing agents as exemplified above or natural gums such as guar gum, gum acacia or gum tragacanth.

Methods for preparing parenterally administrable compositions are apparent to those skilled in the art, and are described in more detail in, for example, Remington's Pharmaceutical Science, 15th ed., Mack Publishing Company, Easton, PA, the entirety of which is hereby incorporated by reference.

Topical formulations may comprise an active ingredient together with one or more acceptable carriers, and optionally any other therapeutic ingredients. Formulations suitable for topical administration include liquid or semi-liquid preparations suitable for penetration through the skin to the site where treatment is required, such as liniments, lotions, creams, ointments or pastes, and drops suitable for administration to the eye, ear or nose.

Drops may comprise sterile aqueous or oily solutions or suspensions. These may be prepared by dissolving the active ingredient in an aqueous solution of a bactericidal and/or fungicidal agent and/or any other suitable preservative, and optionally including a surface active agent. The resulting solution may then be clarified by filtration, transferred to a suitable container and sterilised. Sterilisation may be achieved by autoclaving or maintaining at 90° C. to 100° C. for half an hour, or by filtration, followed by transfer to a container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions include those suitable for application to the skin or eye. An eye lotion may comprise a sterile aqueous solution optionally containing a bactericide and may be prepared by methods similar to those described above in relation to the preparation of drops. Lotions or liniments for application to the skin may also include an agent to hasten drying and to cool the skin, such as an alcohol or acetone, and/or a moisturiser such as glycerol, or oil such as olive oil.

Creams, ointments or pastes are typically semi-solid formulations of the active ingredient for external application. They may be made by mixing the active ingredient in finely divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol, such as propylene glycol or macrogols.

The composition may incorporate any suitable surfactant such as an anionic, cationic or non-ionic surfactant, such as sorbitan esters or polyoxyethylene derivatives thereof. Suspending agents such as natural gums, cellulose derivatives or inorganic materials such as salicaceous silicas, and other ingredients such as lanolin, may also be included.

In some embodiments the compositions are administered in the form of suppositories suitable for rectal administration of the compounds of formulae (I) or (II). These compositions are prepared by mixing the compound of formulae (I) or (II) with a suitable non-irritating excipient which is solid at ordinary temperatures but liquid at the rectal temperature and will therefore melt in the rectum to release the compound of formulae (I) or (II). Such materials include cocoa butter, glycerinated gelatin, hydrogenated vegetable oils, mixtures of polyethylene glycols of various molecular weights and fatty acid esters of polyethylene glycol.

The compositions may also be administered or delivered to target cells in the form of liposomes. Liposomes are generally derived from phospholipids or other lipid substances and are formed by mono- or multi-lamellar hydrated liquid crystals that are dispersed in an aqueous medium. Specific examples of liposomes used in administering or delivering a composition to target cells are synthetic cholesterol (Sigma), the phospholipid 1,2-distearoyl-sn-glycero-3-phosphocholine (DSPC, Avanti Polar Lipids), the PEG lipid 3-N-[(-methoxy poly(ethylene glycol)2000)carbamoyl]-1,2-dimyrestyloxy-propylamine (PEG-cDMA), and the cationic lipid 1,2-di-o-octadecenyl-3-(N.N-dimethyl)aminopropane (DODMA) or 1,2-dilinoleyloxy-3-(N,N-dimethylaminopropane (DLinDMA) in the molar ratios 55:20:10:15 or 48:20:2:30, respectively, PEG-CDMA, DODMA and DLinDMA. The liposome may be constructed from 1,2-distearoyl-sn-glycero-3-phosphoethanolamine-N-[methoxy(polyethyleneglycol)-2000] (DSPE PEG2000) and phosphatidylcholine derived from soy and hydrogenated between 50-100%, for example Soy PC-75 or Soy PC-100. Differing MW PEG's may be used and covalently bound with various specific targeting agents such as glutathione, RGD peptides or other recognized liposome targeting agents. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this, specific reference is made to: Prescott, Ed., Methods in Cell Biology, Volume XIV, Academic Press, New York, N Y. (1976), p. 33 et seq., the contents of which is incorporated herein by reference.

The compositions may also be administered in the form of microparticles or nanoparticles. Biodegradable microparticles formed from polylactide (PLA), polylactide-co-glycolide (PLGA), and epsilon-caprolactone (¿-caprolactone) have been extensively used as drug carriers to increase plasma half-life and thereby prolong efficacy (R. Kumar, M. 2000. J. Pharm. Pharmaceut. Sci. 3 (2) 234-258). Microparticles have been formulated for the delivery of a range of drug candidates including vaccines, antibiotics, and DNA. Moreover, these formulations have been developed for various delivery routes including parenteral subcutaneous injection, intravenous injection and inhalation.

The compositions may incorporate a controlled release matrix that is composed of sucrose acetate isobutyrate (SAIB) and an organic solvent or organic solvents mixture. Polymer additives may be added to the vehicle as a release modifier to further increase the viscosity and slow down the release rate. SAIB is a well-known food additive. It is a very hydrophobic, fully esterified sucrose derivative, at a nominal ratio of six isobutyrate to two acetate groups. As a mixed ester, SAIB does not crystallise but rather exists as a clear viscous liquid. Mixing SAIB with a pharmaceutically acceptable organic solvent, such as ethanol or benzyl alcohol decreases the viscosity of the mixture sufficiently to allow for injection. An active pharmaceutical ingredient may be added to the SAIB delivery vehicle to form SAIB solution or suspension formulations. When the formulation is injected subcutaneous!y. the solvent differs from the matrix allowing the SAIB-drug or SAIB-drug-polymer mixtures to set up as an in situ forming depot.

For administration via inhalation the compound of formulae (I) or (II) may be provided in the form of a dry powder and delivered by a dry powder inhaler (DPI). The dry powder may comprise a bulking agent, for example lactose. DPIs are well known to those skilled in the art. The compound of formulae (I) or (II) may alternatively be delivered in the form of an aerosol. For example, a mixture of the compound of formulae (I) or (II) and a lubricating agent may be dispersed in a propellant and placed into an appropriate metered-dose inhaler for delivery intranasally or via oral inhalation. When the compound of formulae (I) or (II) is administered via inhalation routes the methods of the invention may further comprise administration of a bronchodilator so as to increase airflow to the lungs of the subject. Non-limiting examples of bronchodilators include salbutamol, pirbuterol, terbutaline, clenbuterol, salmeterol, bambuterol and indacaterol. When the compound of formulae (I) or (II) is administered with a bronchodilator each component may be administered at the same time, or sequentially in any order, or at different times so as to provide the desired effect. Alternatively, the components may be formulated together in a single dosage unit.

For the purposes of the present disclosure, compounds and compositions may be administered to subjects either therapeutically or preventively. In a therapeutic application compositions are administered to a patient already suffering from fibrosis or a fibrotic disorder in an amount sufficient to cure, or at least partially arrest the fibrosis or fibrotic disorder and its complications. The composition should provide a quantity of the compound sufficient to effectively treat the subject.

The therapeutically effective amount for any particular subject will depend upon a variety of factors including: the fibrosis or fibrotic disorder being treated and the severity thereof; the activity of the compound administered; the composition in which the compound is present; the age, body weight, general health, sex and diet of the subject; the time of administration; the route of administration; the rate of sequestration of the compound; the duration of the treatment; drugs used in combination or coincidental with the compound, together with other related factors well known in medicine.

One skilled in the art would be able, by routine experimentation, to determine an effective, non-toxic amount of a compound that would be required to achieve the desired outcome.

Generally, an effective dosage is expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage may be up to about 500 mg/m². Generally, an effective dosage is expected to be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

It will be apparent to one of ordinary skill in the art that the optimal quantity and spacing of individual dosages will be determined by the nature and extent of the fibrotic disorder being treated, the form, route and site of administration, and the nature of the particular individual being treated. Also, such optimum conditions can be determined by conventional techniques.

The compounds of formula (I) may be used alone in the treatment of fibrotic disorders, or alternatively in combination therapy with other therapeutic agents, such as for example anti-inflammatory compounds.

The term “combination therapy” is intended to embrace administration of multiple therapeutic agents in a sequential manner in a regimen that will provide beneficial effects and is intended to embrace administration of these agents in either a single formulation or in separate formulations.

Combination therapy may involve the active agents being administered together, sequentially, or spaced apart as appropriate in each case. Combinations of active agents including compounds of the disclosure may be synergistic.

The co-administration of compounds of the formulae (I) and (II) with other therapeutic agent(s) may be effected by a compound of the formulae (I) or (II) being in the same unit dose form as the other therapeutic agent(s), or the compound of the formulae (I) or (II) and the other therapeutic agent(s) may be present in individual and discrete unit dosage forms that are administered sequentially, at the same, or at a similar time. Sequential administration may be in any order as required, and may require an ongoing physiological effect of the first or initial agent to be current when the second or later agent is administered, especially where a cumulative or synergistic effect is desired. When administered separately, it may be preferred for the compound of formulae (I) or (II) and the other agent to be administered by the same route of administration, although it is not necessary for this to be so.

In accordance with various embodiments of the present disclosure one or more compounds of formulae (I) or (II) may be included in combination therapy with surgery.

### EXAMPLES

The present disclosure is further described below by reference to the following non-limiting examples.

### Example 1—Synthesis of Compounds

Compound 7 was prepared according to Scheme 2:

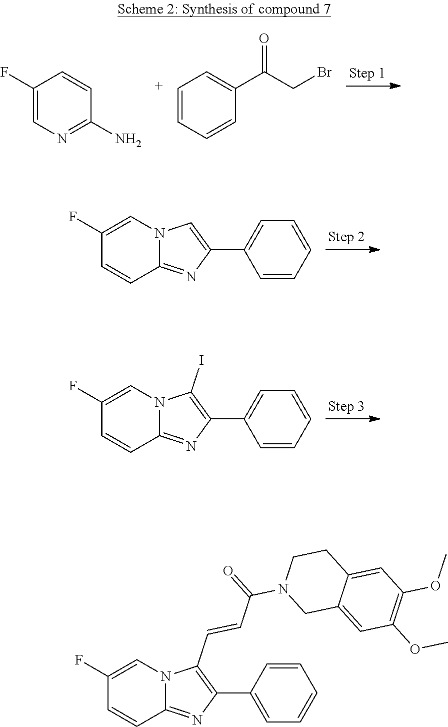 [SMILES: Nc1ccc(F)cn1 ; O=C(CBr)c1ccccc1 ; Fc1ccc2nc(-c3ccccc3)cn2c1 ; Fc1ccc2nc(-c3ccccc3)c(I)n2c1 ; COc1cc2c(cc1OC)CN(C(=O)/C=C/c1c(-c3ccccc3)nc3ccc(F)cn13)CC2]

### Step 1:

To a solution of 2-amino-5-fluoropyridine (1.0 g, 8.9 mmol) in EtOH (8 ml) was added 2-bromo-1-phenyl-ethanone (1.5 g, 7.6 mmol) and NaHCO₃ (0.97 g, 11.6 mmol). The resulting mixture was warmed to 60° C. and stirred overnight. Upon cooling to room temperature, the reaction mixture was diluted with water. The resulting solid was collected by filtration to afford 1.4 g (88%) of 6-fluoro-2-phenylimidazo[1,2-a]pyridine. LCMS (MSMS): m/z: 212.8.

### Step 2:

To a solution of 6-fluoro-2-phenylimidazo[1,2-a]pyridine (0.39 g, 1.8 mmol) in DMF (4 ml) was added N-iodosuccinimide (0.50 g, 2.2 mmol) at room temperature. After 4 hours, the reaction mixture was diluted with water. The resulting solid was collected via filtration to give 0.59 g of the crude product, which was flushed through a pad of silica gel using dichloromethane. Concentration of the eluent afforded 6-fluoro-3-iodo-2-phenylimidazo[1,2-a]pyridine, which was used without further purification.

### Step 3:

To a solution of 6-fluoro-3-iodo-2-phenylimidazo[1,2-a]pyridine (96 mg, 0.3 mmol) and 1-(6,7-dimethoxy-3,4-dihydro-1H-isoquinolin-2-yl)prop-2-en-1-one (84 mg, 0.4 mmol) in DMF (1.5 ml) was added tetrabutyl ammonium chloride (94 mg, 0.4 mmol), K₂CO₃ (78 mg, 0.6 mmol) and Pd(OAc)₂ (20 mg) at room temperature. The resultant mixture was warmed to 100° C. and stirred overnight. Upon cooling to room temperature, the reaction mixture was diluted with water and ethyl acetate. The layers were separated and the aqueous phase extracted with ethyl acetate (2×). The combined organics were dried (MgSO₄), filtered and concentrated to give the crude product. Purification by silica gel flash column chromatography (5-100% ethyl acetate/hexane) afforded (2E)-1-(6,7-dimethoxy-3,4-dihydro-1H-isoquinolin-2-yl)-3-{6-fluoro-2-phenylimidazo[1,2-a]pyridin-3-yl}prop-2-en-1-one as a yellow solid. LCMS (MSMS): m/z: 458.0.

The following compounds were prepared in an analogous manner using the appropriate intermediates:
(2E)-1-(6,7-dimethoxy-3,4-dihydro-1H-isoquinolin-2-yl)-3-{8-fluoro-2-phenylimidazo[1,2-a]pyridin-3-yl}prop-2-en-1-one (compound 4); LCMS (MSMS): m/z: 458.0.
(2E)-1-(4-methoxypiperidin-1-yl)-3-{2-phenylimidazo[1,2-a]pyridin-3-yl}prop-2-en-1-one (compound 3); LCMS (MSMS): m/z: 361.3.
(2E)-1-(1,3-dihydroisoindol-2-yl)-3-{2-phenylimidazo[1,2-a]pyridin-3-yl}prop-2-en-1-one (compound 2); LCMS (MSMS): m/z: 365.3.

2E)-3-[2-(4-chlorophenyl) imidazo[1,2-a]pyridin-3-yl]-1-(6,7-dimethoxy-3,4-dihydro-1H-isoquinolin-2-yl)prop-2-en-1-one (compound 6); LCMS (MSMS): m/z: 473.8.
(2E)-1-(6,7-dimethoxy-3,4-dihydro-1H-isoquinolin-2-yl)-3-[2-(4-fluorophenyl) imidazo[1,2-a]pyridin-3-yl]prop-2-en-1-one (compound 5); LCMS (MSMS): m/z: 458.0.

Precursor compounds in scheme 2 may be synthesised as follows.

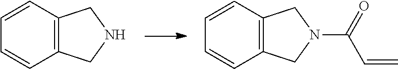

### 1-(1,3-dihydroisoindol-2-yl)prop-2-en-1-one

To a mixture of isoindoline (0.50 g, 4.2 mmol) in DCM (20 ml) is added triethylamine (25.2 mmol, 3.5 ml) at −78° C. After ten minutes, a solution of acryloyl chloride (0.46 g, 5.0 mmol) in DCM (1 ml) is added in a dropwise manner. After 0.5 h the reaction is concentrated and diluted with DCM and water. The layers are separated and the aqueous phase is extracted with DCM (2×). The combined organics are washed with brine and dried (MgSO₄). Filtration and concentration gives the crude product which is purified via flash column chromatography (0-10% ethyl acetate/DCM) to afford the desired product as a light orange solid.

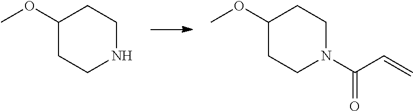

### 1-(4-methoxypiperidin-1-yl)prop-2-en-1-one

A mixture of 4-methoxypiperidine (0.50 g, 4.3 mmol) and triethylamine (0.84 ml, 6.1 mmol) in DCM (5 ml) is cooled to −10° C. Acryloyl chloride (0.47 g, 5.2 mmol) is added, dropwise, as a DCM solution (1 ml). After ten minutes the cooling bath is removed and the reaction is stirred at ambient temperature for 2 hours. The reaction is poured into water and DCM and the layers are separated. The aqueous phase is extracted with DCM (2×). The combined organics are washed with brine, dried (MgSO₄), filtered and concentrated to give the crude product. Purification via flash column chromatography (0-20% ethyl acetate/DCM) gives the desired product.

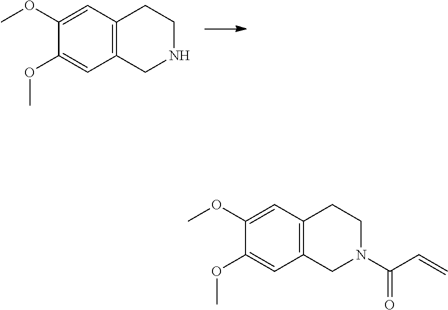 [SMILES: COc1cc2c(cc1OC)CNCC2 ; C=CC(=O)N1CCc2cc(OC)c(OC)cc2C1]

### 1-(6,7-dimethoxy-3,4-dihydro-1H-isoquinolin-2-yl)prop-2-en-1-one

To a solution of 6,7-dimethoxy-1,2,3,4-tetrahydroisoquinoline (1 g, 5 mmol) in THF (10 ml) are added K₂CO₃ (1.4 g, 10 mmol) and acryloyl chloride (540 mg, 6 mmol) at room temperature. The mixture is heated at 50° C. for 3 hours. The mixture is cooled down and water (15 ml) is added. The mixture is extracted with ethyl acetate (15 mL×3) and the combined organic layer is dried (MgSO₄). The mixture is filtered and the filtrate is concentrated under reduced pressure. The residue is purified by silica gel flash column chromatography with 20% ethyl acetate in Hexane as the mobile phase to afford 1-(6,7-dimethoxy-3,4-dihydro-1H-isoquinolin-2-yl)prop-2-en-1-one as a white solid (1.17 g, 95%). LCMS (ESMS): m/z: 248.3 (M⁺+1).

### Example 2—Assays

Transforming TGF-β regulates the proliferation and differentiation of fibroblasts, which are the primary constituent cells of fibrous tissues. As such, representative compounds of the present disclosure were tested for their ability to inhibit TGF-β.

### Materials

1. TGFβ/SMAD Signaling Pathway SBE Reporter—HEK293 Cell Line BPS Bioscience Catalog #: 60653
2. Human TGFβ1 (BPS Bioscience #90900-1)
3. SIS3 (Sigma #S0447): inhibitor of TGFβ pathway. Prepare stock solution in DMSO.
4. Growth medium: MEM medium (Invitrogen #11095-080)+10% FBS (ATCC #30-2020)+1% non-essential amino acids (Lonza #13-114E)+1 mM Na pyruvate (Lonza #13-115E)+1% Penicillin/Streptomycin (ATCC #30-2300)+400 μg/ml of Geneticin (Invitrogen #11811-031).
5. Assay medium: MEM medium+0.5% FBS+1% non-essential amino acids+1 mM Na pyruvate+1% Pen/Strep
6. 96-well tissue culture-treated white clear-bottom assay plate (Corning #3610)
7. ONE-Step™ Luciferase Assay System (BPS, Cat. #60690)

### Method

SBE reporter-HEK293 cells were harvested from culture in growth medium and seeded at a density of ˜10,000 cells per well into a white clear-bottom 96-well microplate in 100 μl of assay medium. The plate containing cells were incubated at 37° C. in a CO₂ incubator. 24 hours after seeding, cells were washed with 100 μL of assay buffer and treated with three fold serial dilution of compounds in 50 μl assay medium. Cells were incubated at 37° C. in a CO₂ incubator for 4 hours. For control wells 50 μl assay medium was added with no inhibitor. Each treatment was done in triplicate. After 4 hours of incubation, 5 μl of human TGFβ1 in assay medium was added to cells (final TGFβ1 concentration=20 ng/ml). 5 μl of assay medium was added to the unstimulated control wells (for determining the basal activity). The plate with cells was incubated at 37° C. in a CO₂ incubator overnight (˜18 hours). After 18 hours of incubation Luciferase assay was performed using ONE-Step™ Luciferase Assay System according to the protocol provided: 50 μl of ONE-Step™ Luciferase reagent was added per well and shook at room temperature for ˜10 minutes. Luminescence was measured using a luminometer.

### Results

The results are shown below in Table 1

| Compound | IC₅₀ (μm) |
|---|---|
| SIS3* | 2.651 |
| 6 | 5.445 |
| 8 | 3.716 |
| 9 | 6.114 |
| 10 | 4.319 |
| 11 | 3.515 |

*comparative compound

Compounds 6, 8, 9, 10 and 11 all showed inhibitory activity against TGF-β.

The citation of any reference herein should not be construed as an admission that such reference is available as prior art to the present application. Further, the reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavour to which this specification relates.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of an two or more of said steps, features, compositions and compounds.

1. A method for treating fibrosis in a subject in need thereof, the method comprising administration to the subject of a therapeutically effective amount of a compound having the following formula (I), or a pharmaceutically acceptable salt or prodrug thereof: 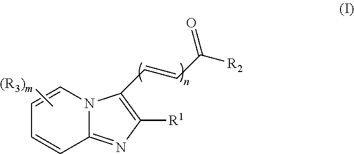
wherein:
R₁ is selected from the group consisting of: C₆₋₁₀aryl, a heteroaryl group having between 5 and 10 ring atoms wherein one or more of the ring atoms are selected from nitrogen, sulfur and oxygen, C₃₋₁₀cycloalkyl and a heterocyclyl group having between 5 and 10 ring atoms wherein one or more of the ring atoms are selected from nitrogen, sulfur and oxygen, each of which is optionally substituted with one or more of the following groups: C₁₋₆alkoxy, C₁₋₆alkyl, CN, halo and C₃₋₁₀cycloalkyl;
R₂ is selected from the group consisting of:
(i) C₆₋₁₀aryl, a heteroaryl group having between 5 and 10 ring atoms wherein one or more of the ring atoms are selected from nitrogen, sulfur and oxygen, and a heterocyclyl group having between 5 and 10 ring atoms wherein one or more of the ring atoms are selected from nitrogen, sulfur and oxygen, each of which is optionally substituted with one or more of the following groups: NR₅R₆, C₁₋₆alkyl, C₁₋₆alkoxy and halo; and
(ii)-X-Y, wherein
X is selected from the group consisting of: a heterocyclyl group having between 5 and 10 ring atoms wherein one or more of the ring atoms are selected from nitrogen, sulfur and oxygen, C₆₋₁₀aryl and a heteroaryl group having between 5 and 10 ring atoms wherein one or more of the ring atoms are selected from nitrogen, sulfur and oxygen; and
Y is selected from the group consisting of: C₆₋₁₀aryl and a heteroaryl group having between 5 and 10 ring atoms wherein one or more of the ring atoms are selected from nitrogen, sulfur and oxygen, and wherein the C₆₋₁₀aryl and heteroaryl groups of Y are optionally substituted with one or more of the following groups: NR₅R₆, C₁₋₆alkyl, C₁₋₆alkoxy and halo;
R₃ is selected from the group consisting of: halo, C₁₋₆ alkoxy, OH, CN, NO₂, —OC(O)R₄, —C(O)R₄, —C(O)NR₅R₆, —C(O)OR₄, —OS(O)₂R₄, NR₅R₆, SR₄, C₁₋₁₀alkyl, C₂₋₁₀alkenyl, C₂₋₁₀alkynyl, C₃₋₁₀cycloalkyl, C₆₋₁₀aryl and a heteroaryl group having between 5 and 10 ring atoms wherein one or more of the ring atoms are selected from nitrogen, sulfur and oxygen;
R₄ is selected from H and C₁₋₆alkyl;
R₅ and R₆ are independently selected from H and C₁₋₆alkyl;
n is 1 or 2; and,
m is an integer between 0 and 4.

2-5. (canceled)

6. The method of claim 1, wherein R₁ is selected from the group consisting of: phenyl, a heteroaryl group having 5 or 6 ring atoms wherein one or more of the ring atoms are selected from nitrogen, sulfur and oxygen and a heterocyclyl group having 5 or 6 ring atoms wherein one or more of the ring atoms are selected from nitrogen, sulfur and oxygen, each of which is optionally substituted with one or more of the following groups: C₁₋₄alkyl, halo and C₃₋₄cycloalkyl.

7-11. (canceled)

12. The method of claim 1, wherein R₁ is selected from the group consisting of: phenyl, a heteroaryl group having 5 or 6 ring atoms wherein between one and three of the ring atoms are selected from nitrogen, sulfur and oxygen and a heterocyclyl group having 6 ring atoms wherein one or two of the ring atoms are selected from nitrogen and oxygen, each of which is optionally substituted with one of the following groups: methyl, chloro, fluoro and cyclopropyl.

13. The method of claim 1, wherein R₁ is selected from the group consisting of:

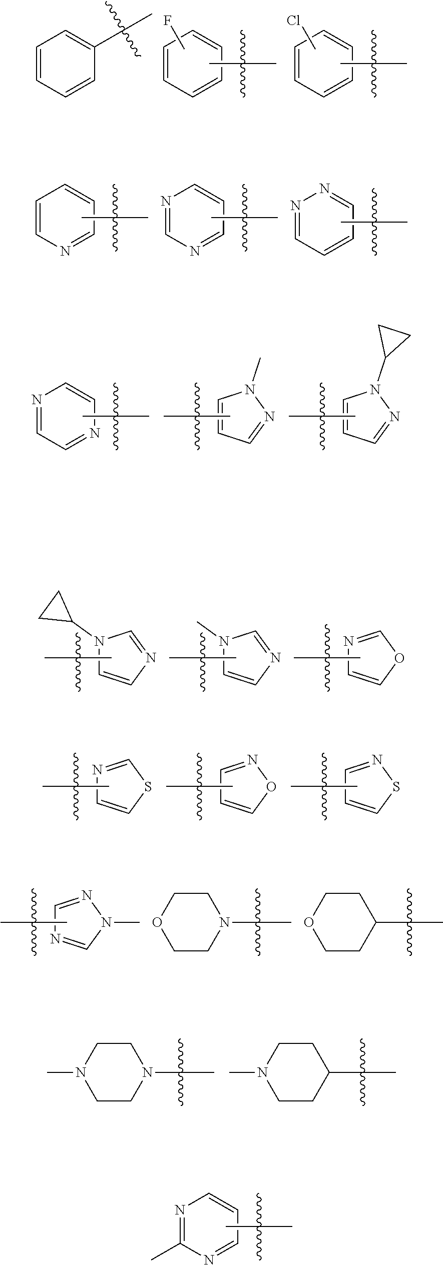

14. (canceled)

15. The method of claim 1, wherein R₂ is selected from the group consisting of:
(i) phenyl, a heteroaryl group having between 5 and 10 ring atoms wherein one or more of the ring atoms are nitrogen, a heterocyclyl group having between 5 and 10 ring atoms wherein one or more of the ring atoms are nitrogen or oxygen, each of which is optionally substituted with one or more of the following groups: NR₅R₆, C₁₋₆alkyl, C₁₋₆alkoxy and halo; and
(ii)-X-Y, wherein
X is selected from the group consisting of: a heterocyclyl group having between 5 and 10 ring atoms wherein one or more of the ring atoms are nitrogen, phenyl and a heteroaryl group having between 5 or 6 ring atoms wherein one or more of the ring atoms are nitrogen; and
Y is selected from the group consisting of: phenyl and a heteroaryl group having 5 or 6 ring atoms wherein one or more of the ring atoms are nitrogen, and wherein the phenyl and heteroaryl groups of Y are optionally substituted with one or more of the following groups: NR₅R₆, C₁₋₆alkyl, C₁₋₆alkoxy and halo.

16-21. (canceled)

22. The method of claim 1, wherein R₂ is selected from the group consisting of:
(i) a heterocyclyl group having between 5 and 10 ring atoms wherein between one and four of the ring atoms are nitrogen or oxygen, each of which is optionally substituted with between one and three of the following groups: NH₂, N(Me)₂, methyl and methoxy, and
(ii)-X-Y, wherein
X is selected from the group consisting of: a heterocyclyl group having 6 ring atoms wherein one or two of the ring atoms are nitrogen; and
Y is selected from the group consisting of: phenyl and a heteroaryl group having 5 or 6 ring atoms wherein one or two of the ring atoms are nitrogen, and wherein the phenyl and heteroaryl groups of Y are optionally substituted with one or two of the following groups: methyl, methoxy, fluoro and chloro.

23. The method of claim 1, wherein R₂ is selected from the group consisting of:

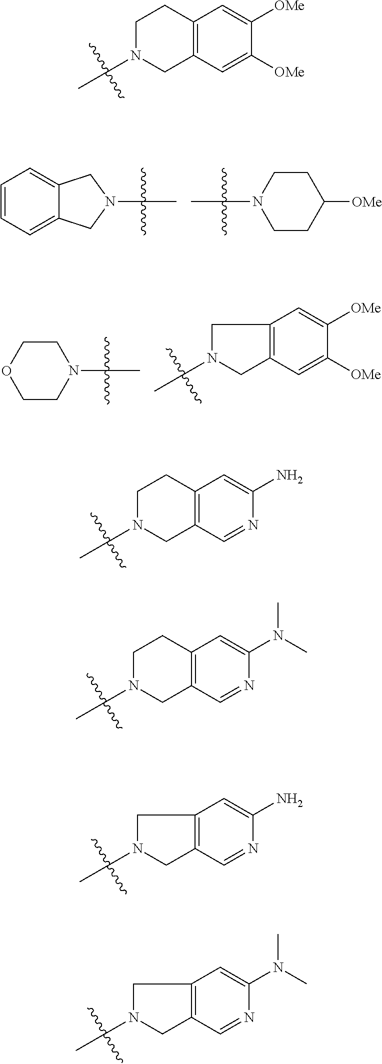

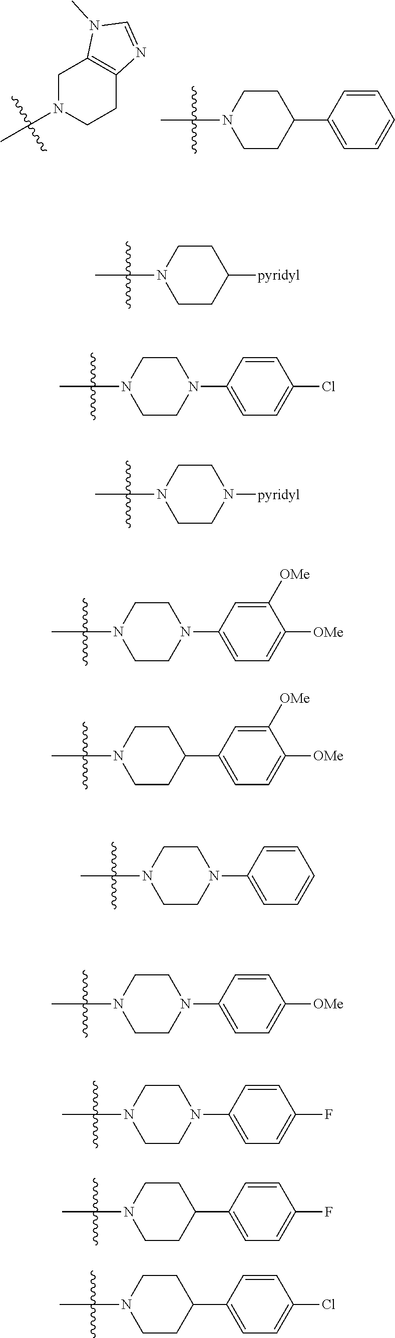

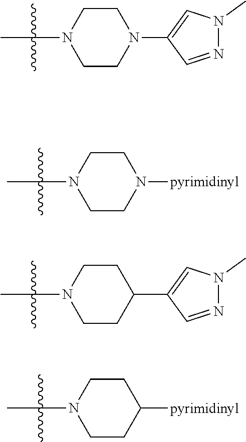

24-25. (canceled)

26. The method of claim 1, wherein R₃ is selected from the group consisting of: halo, C₁₋₆ alkoxy, OH, CN, NR₅R₆, C₁₋₆alkyl, C₂₋₆alkenyl, C₂₋₆alkynyl, C₃₋₆cycloalkyl, C₆₋₁₀aryl and a heteroaryl group having between 5 or 6 ring atoms wherein one or more of the ring atoms are selected from nitrogen, sulfur and oxygen.

27-28. (canceled)

29. The method of claim 1, wherein R₃ is selected from the group consisting of: halo, C₁₋₄ alkoxy, OH, CN, C₁₋₄alkyl, phenyl and a heteroaryl group having between 5 or 6 ring atoms wherein one or more of the ring atoms are selected from nitrogen, sulfur and oxygen.

30-35. (canceled)

36. The method of claim 1, wherein R₄ is H or CH₃.

37. The method of claim 1, wherein R₅ and R₆ are independently selected from H and CH₃.

38. The method of claim 1, wherein n is 1.

39. (canceled)

40. The method of claim 1, wherein m is 0 or 1.

41. The method according to claim 1, wherein the compound of formula (I) is selected from the group consisting of:

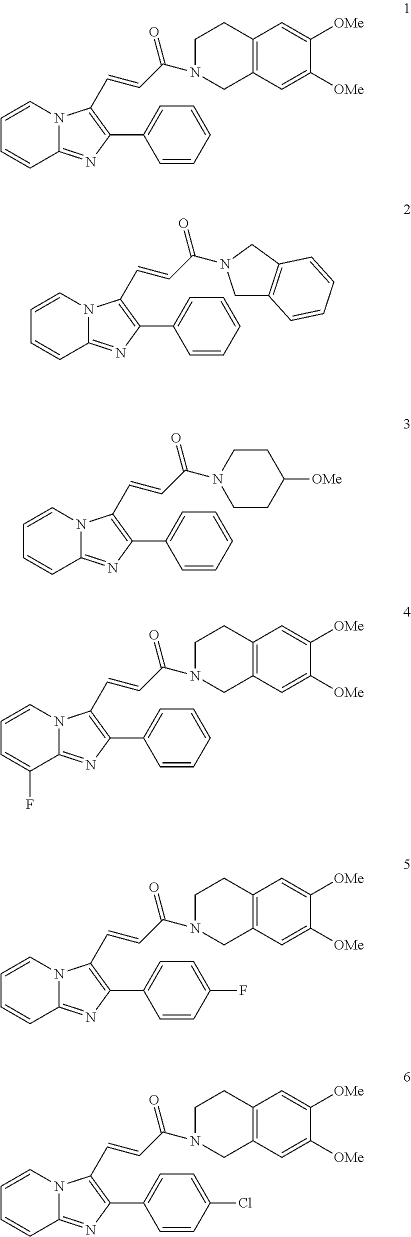 [SMILES: COc1cc2c(cc1OC)CN(C(=O)/C=C/c1c(-c3ccccc3)nc3ccccn13)CC2 ; O=C(/C=C/c1c(-c2ccccc2)nc2ccccn12)N1Cc2ccccc2C1 ; COC1CCN(C(=O)/C=C/c2c(-c3ccccc3)nc3ccccn23)CC1 ; COc1cc2c(cc1OC)CN(C(=O)/C=C/c1c(-c3ccccc3)nc3c(F)cccn13)CC2 ; COc1cc2c(cc1OC)CN(C(=O)/C=C/c1c(-c3ccc(F)cc3)nc3ccccn13)CC2 ; COc1cc2c(cc1OC)CN(C(=O)/C=C/c1c(-c3ccc(Cl)cc3)nc3ccccn13)CC2]

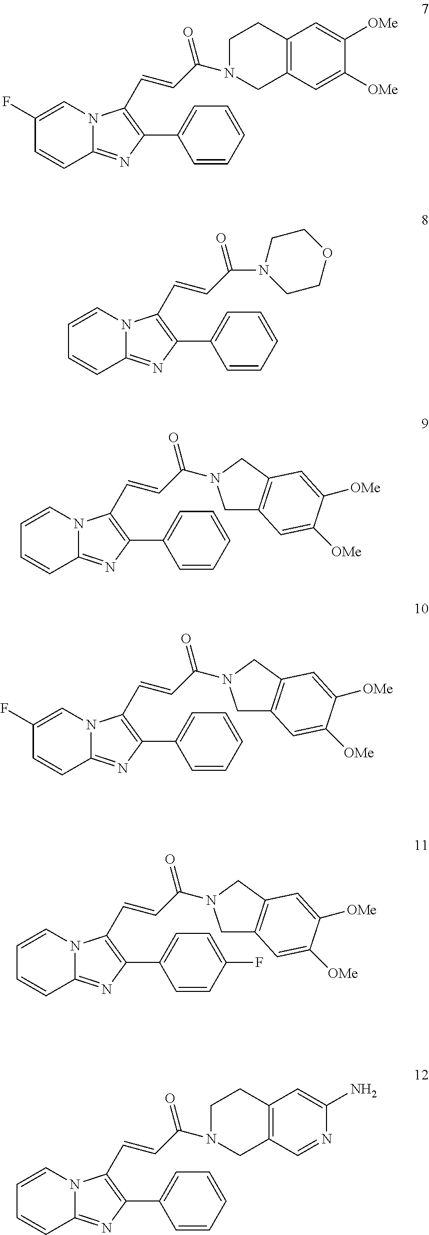 [SMILES: COc1cc2c(cc1OC)CN(C(=O)/C=C/c1c(-c3ccccc3)nc3ccc(F)cn13)CC2 ; O=C(/C=C/c1c(-c2ccccc2)nc2ccccn12)N1CCOCC1 ; COc1cc2c(cc1OC)CN(C(=O)/C=C/c1c(-c3ccccc3)nc3ccccn13)C2 ; COc1cc2c(cc1OC)CN(C(=O)/C=C/c1c(-c3ccccc3)nc3ccc(F)cn13)C2 ; COc1cc2c(cc1OC)CN(C(=O)/C=C/c1c(-c3ccc(F)cc3)nc3ccccn13)C2 ; Nc1cc2c(cn1)CN(C(=O)/C=C/c1c(-c3ccccc3)nc3ccccn13)CC2]

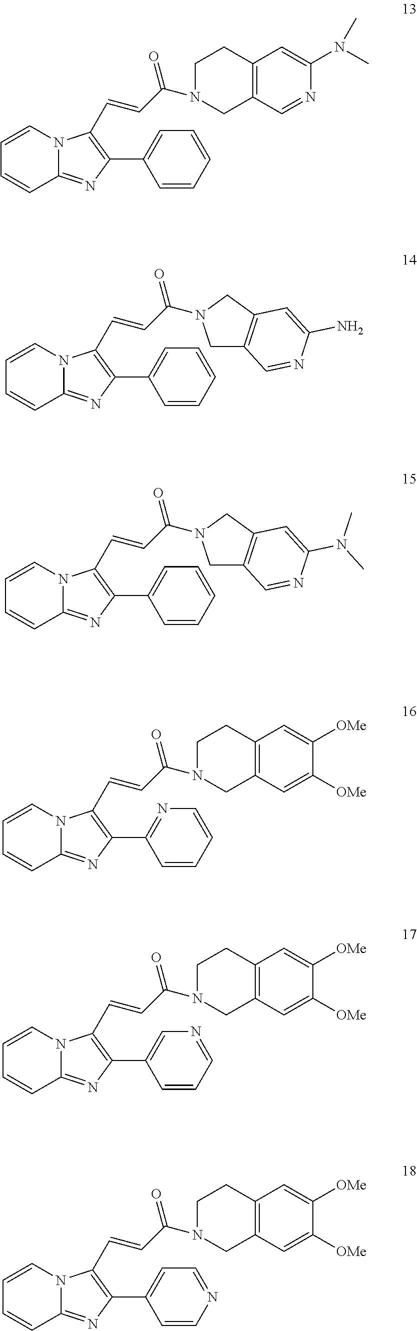 [SMILES: CN(C)c1cc2c(cn1)CN(C(=O)/C=C/c1c(-c3ccccc3)nc3ccccn13)CC2 ; Nc1cc2c(cn1)CN(C(=O)/C=C/c1c(-c3ccccc3)nc3ccccn13)C2 ; CN(C)c1cc2c(cn1)CN(C(=O)/C=C/c1c(-c3ccccc3)nc3ccccn13)C2 ; COc1cc2c(cc1OC)CN(C(=O)/C=C/c1c(-c3ccccn3)nc3ccccn13)CC2 ; COc1cc2c(cc1OC)CN(C(=O)/C=C/c1c(-c3cccnc3)nc3ccccn13)CC2 ; COc1cc2c(cc1OC)CN(C(=O)/C=C/c1c(-c3ccncc3)nc3ccccn13)CC2]

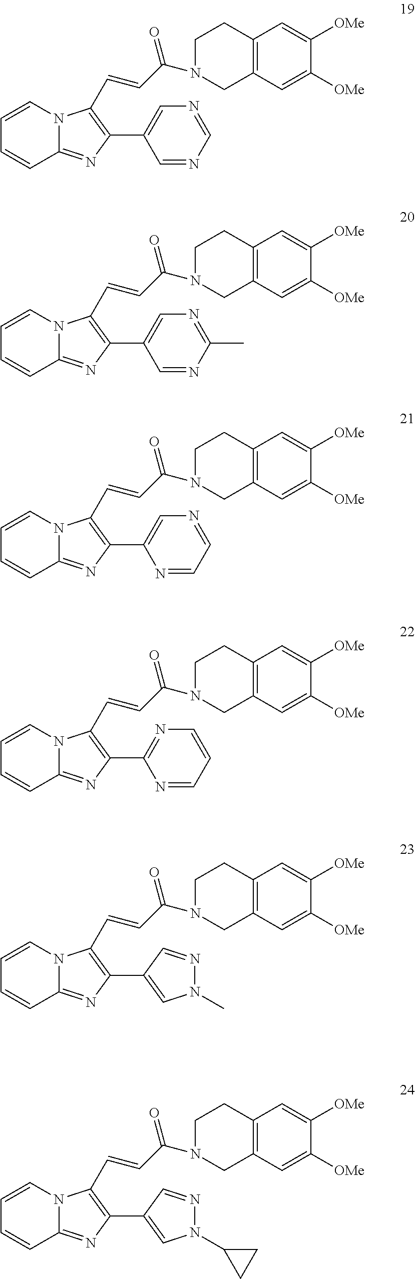 [SMILES: COc1cc2c(cc1OC)CN(C(=O)/C=C/c1c(-c3cncnc3)nc3ccccn13)CC2 ; COc1cc2c(cc1OC)CN(C(=O)/C=C/c1c(-c3cnc(C)nc3)nc3ccccn13)CC2 ; COc1cc2c(cc1OC)CN(C(=O)/C=C/c1c(-c3cnccn3)nc3ccccn13)CC2 ; COc1cc2c(cc1OC)CN(C(=O)/C=C/c1c(-c3ncccn3)nc3ccccn13)CC2 ; COc1cc2c(cc1OC)CN(C(=O)/C=C/c1c(-c3cnn(C)c3)nc3ccccn13)CC2 ; COc1cc2c(cc1OC)CN(C(=O)/C=C/c1c(-c3cnn(C4CC4)c3)nc3ccccn13)CC2]

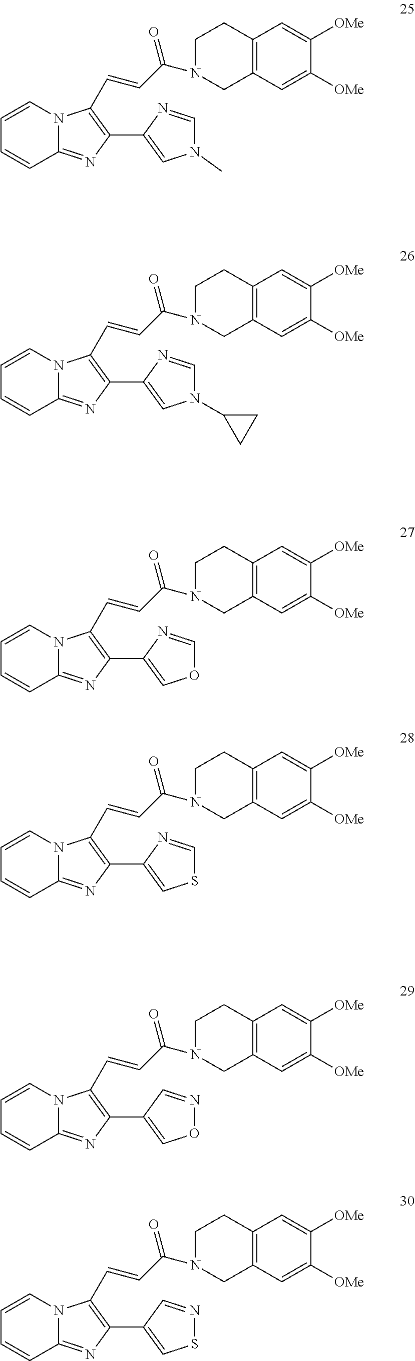 [SMILES: COc1cc2c(cc1OC)CN(C(=O)/C=C/c1c(-c3cn(C)cn3)nc3ccccn13)CC2 ; COc1cc2c(cc1OC)CN(C(=O)/C=C/c1c(-c3cn(C4CC4)cn3)nc3ccccn13)CC2 ; COc1cc2c(cc1OC)CN(C(=O)/C=C/c1c(-c3cocn3)nc3ccccn13)CC2 ; COc1cc2c(cc1OC)CN(C(=O)/C=C/c1c(-c3cscn3)nc3ccccn13)CC2 ; COc1cc2c(cc1OC)CN(C(=O)/C=C/c1c(-c3cnoc3)nc3ccccn13)CC2 ; COc1cc2c(cc1OC)CN(C(=O)/C=C/c1c(-c3cnsc3)nc3ccccn13)CC2]

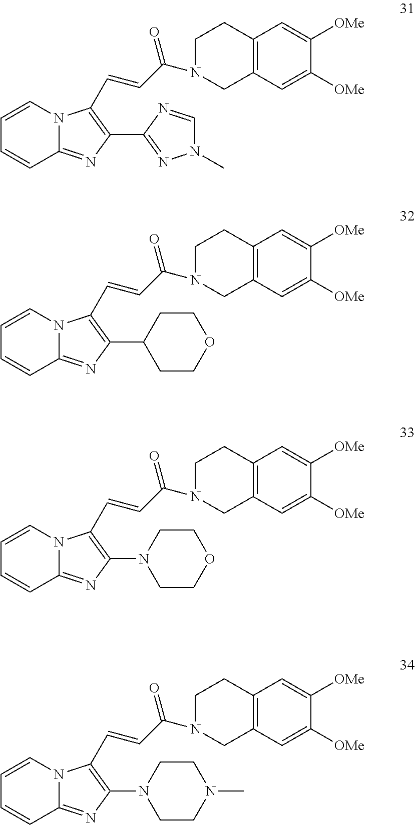 [SMILES: COc1cc2c(cc1OC)CN(C(=O)/C=C/c1c(-c3ncn(C)n3)nc3ccccn13)CC2 ; COc1cc2c(cc1OC)CN(C(=O)/C=C/c1c(C3CCOCC3)nc3ccccn13)CC2 ; COc1cc2c(cc1OC)CN(C(=O)/C=C/c1c(N3CCOCC3)nc3ccccn13)CC2 ; COc1cc2c(cc1OC)CN(C(=O)/C=C/c1c(N3CCN(C)CC3)nc3ccccn13)CC2]

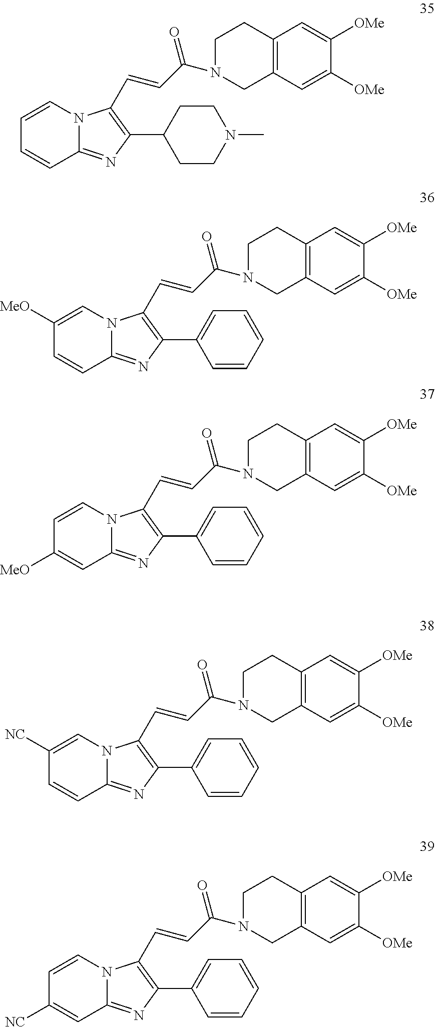 [SMILES: COc1cc2c(cc1OC)CN(C(=O)/C=C/c1c(C3CCN(C)CC3)nc3ccccn13)CC2 ; COc1ccc2nc(-c3ccccc3)c(/C=C/C(=O)N3CCc4cc(OC)c(OC)cc4C3)n2c1 ; COc1ccn2c(/C=C/C(=O)N3CCc4cc(OC)c(OC)cc4C3)c(-c3ccccc3)nc2c1 ; COc1cc2c(cc1OC)CN(C(=O)/C=C/c1c(-c3ccccc3)nc3ccc(C#N)cn13)CC2 ; COc1cc2c(cc1OC)CN(C(=O)/C=C/c1c(-c3ccccc3)nc3cc(C#N)ccn13)CC2]

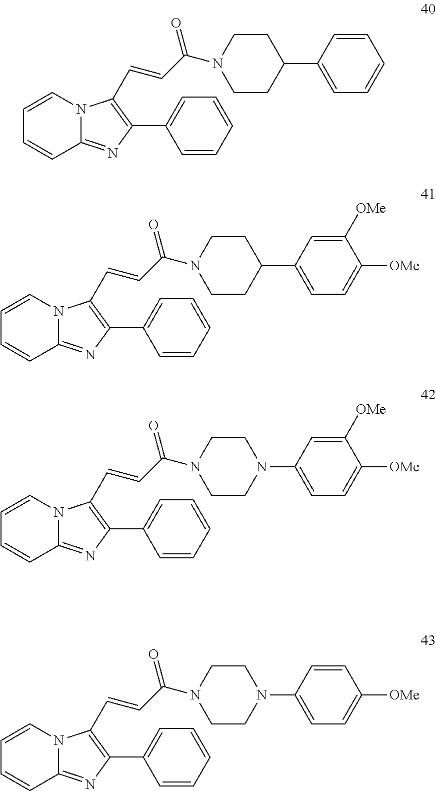 [SMILES: O=C(/C=C/c1c(-c2ccccc2)nc2ccccn12)N1CCC(c2ccccc2)CC1 ; COc1ccc(C2CCN(C(=O)/C=C/c3c(-c4ccccc4)nc4ccccn34)CC2)cc1OC ; COc1ccc(N2CCN(C(=O)/C=C/c3c(-c4ccccc4)nc4ccccn34)CC2)cc1OC ; COc1ccc(N2CCN(C(=O)/C=C/c3c(-c4ccccc4)nc4ccccn34)CC2)cc1]

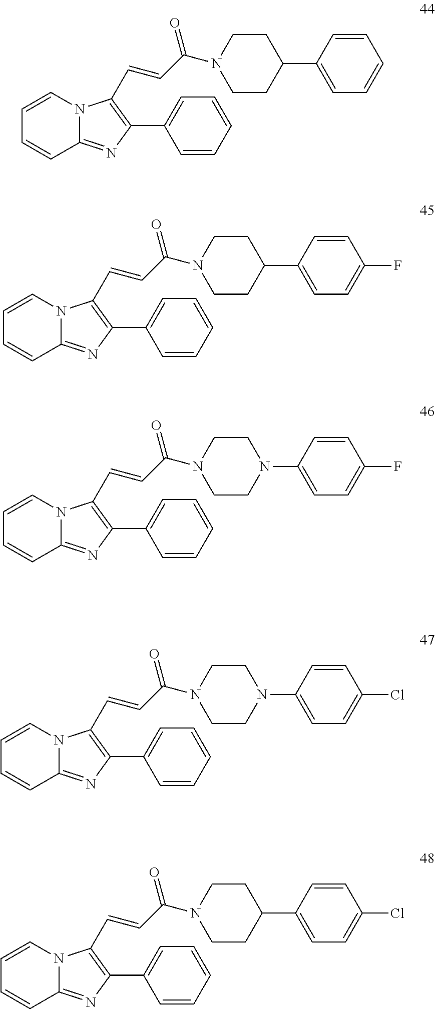 [SMILES: O=C(/C=C/c1c(-c2ccccc2)nc2ccccn12)N1CCC(c2ccccc2)CC1 ; O=C(/C=C/c1c(-c2ccccc2)nc2ccccn12)N1CCC(c2ccc(F)cc2)CC1 ; O=C(/C=C/c1c(-c2ccccc2)nc2ccccn12)N1CCN(c2ccc(F)cc2)CC1 ; O=C(/C=C/c1c(-c2ccccc2)nc2ccccn12)N1CCN(c2ccc(Cl)cc2)CC1 ; O=C(/C=C/c1c(-c2ccccc2)nc2ccccn12)N1CCC(c2ccc(Cl)cc2)CC1]

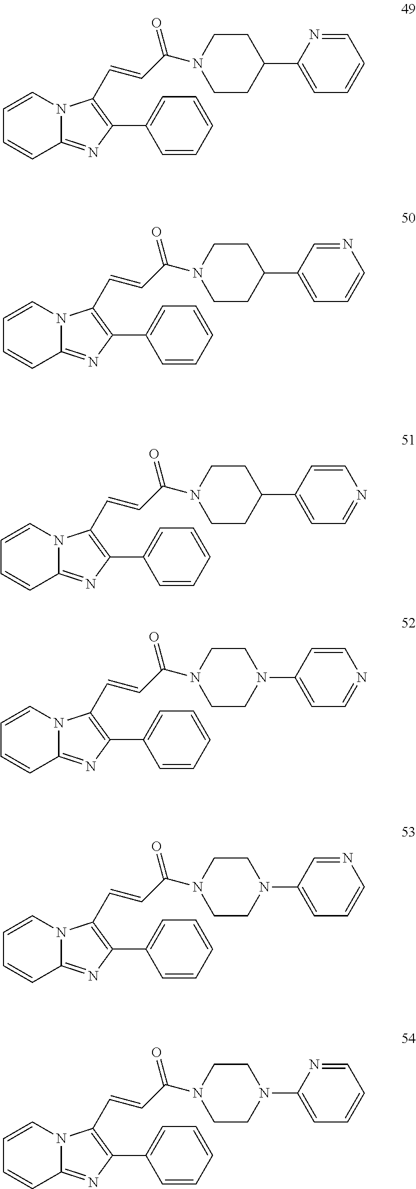 [SMILES: O=C(/C=C/c1c(-c2ccccc2)nc2ccccn12)N1CCC(c2ccccn2)CC1 ; O=C(/C=C/c1c(-c2ccccc2)nc2ccccn12)N1CCC(c2cccnc2)CC1 ; O=C(/C=C/c1c(-c2ccccc2)nc2ccccn12)N1CCC(c2ccncc2)CC1 ; O=C(/C=C/c1c(-c2ccccc2)nc2ccccn12)N1CCN(c2ccncc2)CC1 ; O=C(/C=C/c1c(-c2ccccc2)nc2ccccn12)N1CCN(c2cccnc2)CC1 ; O=C(/C=C/c1c(-c2ccccc2)nc2ccccn12)N1CCN(c2ccccn2)CC1]

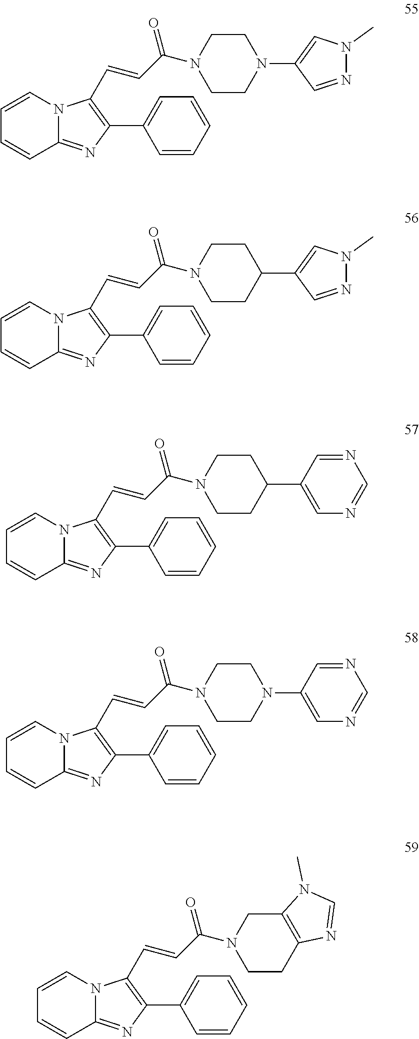 [SMILES: Cn1cc(N2CCN(C(=O)/C=C/c3c(-c4ccccc4)nc4ccccn34)CC2)cn1 ; Cn1cc(C2CCN(C(=O)/C=C/c3c(-c4ccccc4)nc4ccccn34)CC2)cn1 ; O=C(/C=C/c1c(-c2ccccc2)nc2ccccn12)N1CCC(c2cncnc2)CC1 ; O=C(/C=C/c1c(-c2ccccc2)nc2ccccn12)N1CCN(c2cncnc2)CC1 ; Cn1cnc2c1CN(C(=O)/C=C/c1c(-c3ccccc3)nc3ccccn13)CC2]

42. The method of claim 1, wherein the fibrosis is pulmonary fibrosis, fibrosis resulting from radiation, renal fibrosis, cardiac fibrosis, liver fibrosis, arthrofibrosis, nephrogenic systemic fibrosis, retroperitoneal fibrosis, scleroderma/systemic sclerosis, myelofibrosis or mediastinal fibrosis, or the fibrosis is associated with arterial stiffness, Crohn's disease, Dupuytren's contracture, keloids, Peyronie's disease or adhesive capsulitis.

43-45. (canceled)

46. The method of claim 1, wherein the fibrosis is caused by, associated with, or otherwise related to a coronavirus infection in the subject.

47-49. (canceled)

50. The method of claim 1, wherein the compound of formula (I) is administered together with one or more antiviral compounds or anti-inflammatory compounds.

51. A method for preventing or minimising fibrosis or lung injury in a subject, wherein the subject is infected with a coronavirus, or at risk of becoming infected with a coronavirus, the method comprising administering to the subject an effective amount of a compound of formula (I) as defined in claim 1.

52-63. (canceled)

64. A method for minimising the severity of a coronavirus infection in a subject, the method comprising administering to the subject an effective amount of a compound of formula (I) as defined in claim 1.

65-81. (canceled)
